(19) United States
(12) Patent Application Publication
(10) Pub. No.: US 2015/0328168 A1
(43) Pub. Date: Nov. 19, 2015

(54) ORAL FORM, COMPRISING IMMEDIATE-RELEASE COATED PARTICLES OF AT LEAST ONE ACTIVE COMPOUND THAT ARE GRINDING-RESISTANT

(71) Applicant: Flamel Ireland Limited, Dublin (IE)

(72) Inventors: Anne-Sophie DAVIAUD-VENET, Saint Genis Laval (FR); Catherine CASTAN, Orlienas (FR)

(73) Assignee: Flamel Ireland Limited, Dublin (IE)

(21) Appl. No.: 14/651,468

(22) PCT Filed: Dec. 12, 2013
(86) PCT No.: PCT/IB2013/060843

§ 371 Date: Jun. 11, 2015

Related U.S. Application Data
(60) Provisional application No. 61/736,818, filed on Dec. 13, 2012.

(30) Foreign Application Priority Data
Dec. 13, 2012 (FR) 1261991

(51) Int. Cl. A61K 9/50; A61K 9/00; A61K 31/485; A61K 9/20; A61K 9/48

(52) U.S. Cl. CPC A61K 9/5047; A61K 9/2077; A61K 9/4808; A61K 9/2095; A61K 31/485; A61K 9/4833; A61K 9/0053

(57) ABSTRACT

An oral dosage form for the immediate release of at least one active compound, comprising coated particles consisting of a non-monocrystalline core containing said active compound and coated with at least one coating layer, said coating layer comprising (A) at least 15% by weight of water-insoluble polymer and (B) at least 40% by weight of polymer soluble in a 0.1N hydrochloric acid solution, the weight ratio polymer B/polymer A being comprised between 85/15 and 50/50, said coating layer representing at least 30% by weight of the total weight of said coated particles.

The present invention aims to provide a solid oral dosage form, enabling on the one hand the immediate release of the active compound in a 0.1N hydrochloric acid solution, representative of a gastric medium and, on the other hand, resisting most crushing ways used to crush the solid oral dosage form with a view to obtaining the active compound in the dosage form as a fine powder and facilitating its diverted use.

Generally, solid oral dosage forms such as capsules or tablets present insufficient resistance to extraction of the active compound they contain and can therefore be misused.

Thus, a number of medicines exist, for instance psychotropic medications and narcotics, which are subject to abuse: their use is diverted from the authorized indication in order to obtain a rapid euphoric effect, similar to that obtained with illegal drugs.

It is generally sufficient to crush the contents of the capsule or the tablet in order to make the active compound available. Then, the obtained fine powder can be inhaled or dissolved and extracted to prepare an injectable product.

In order to prevent these fraudulent acts, it is essential to have solid oral pharmaceutical forms which make difficult any use other than therapeutic uses officially approved by the competent public health authorities.

Technical solutions aiming to reduce misuse have already been proposed for dosage forms for the modified release of active compound. Thus, the document WO 2007/054378 describes solid oral pharmaceutical forms made up of crush-resistant particles of active compound comprising a modified-release coating, said particles being optionally combined with a viscosity modifying agent or a sequestering agent.

Unlike the forms for the modified release of active ingredient, i.e. allowing delayed, sustained or sequential release of the active compound, the formulations for the immediate release of active compound must release most of the active compound they contain within a relatively short time. The immediate release solid oral dosage form disintegrates very rapidly, in order to release the active compound as quickly as possible.

Various dosage forms for the immediate release of active ingredient have already been described. For example, the document US 2006/0078614 proposes compositions comprising particles coated with a membrane in order to mask the taste of the active compound they contain and enabling the rapid release of almost all of their contents in the stomach. The document EP 1 491 184 describes immediate release tablets which are film-coated with a coating for masking the taste of active ingredient contained therein. More particularly, the document US 2012/0093938 details the composition of orally dispersible tablets for immediate release of diphenhydramine and its salts, the active ingredient particles contained in the tablets being optionally film-coated with a coating for masking taste of active ingredient contained therein. The document US 2012/0082729 discloses an oral dosage form obtained by compression and rapidly dissolving in the mouth, comprising active compound microparticles which can be coated with a coating for masking taste of active ingredient contained therein.

The document US 2013/0303494 claims oral dosage forms for immediate release of active principle, comprising a gelling agent to prevent parenteral or nasal administration under any attempts to misuse said dosage forms. In addition, there are Oxecta® tablets which are commercialized in United States by the company Pfizer. These tablets for immediate release of oxycodone hydrochloride aim to deter from misusing. When crushed, the Oxecta® tablets and oxycodone hydrochloride contained therein are reduced into fine powder. When this fine powder comes into contact with water, either used as extracting solvent by abuser or present around nasal mucous membranes, a gel is formed that prevents Oxecta® tablets misuse.

For obvious reasons, these oral dosage forms for immediate release of active compound and aiming to prevent misuse can be improved. For example, if the conversion of the oral dosage form and of the active compound contained thereof into fine powder could not be done, extraction of the active compound contained in oral dosage form could be more difficult.

The present invention solves this problem.

The inventors have discovered that coated particles comprising the active compound and having a specific coating composition and structure, as described hereafter, are resistant to crushing. They enable providing, according a relatively cheap and rapid industrial process, solid oral dosage forms preventing misuse of the active compound they contain, without affecting the immediate release of said active compound in a 0.1N hydrochloric acid solution.

Thus, according to a first of its aspects, the invention relates to an oral dosage form, in particular a multiparticulate oral dosage form, for the immediate release of at least one active compound, comprising coated particles, each of said particles consisting of a non-monocrystalline core containing said active compound,

said core being coated with at least one coating layer comprising:

(A) at least 15% by weight of polymer chosen from ethylcellulose, cellulose acetate, cellulose acetate butyrate, ammonio (meth)acrylate copolymers, polymers and copolymers of (meth)acrylic acid esters, polyvinyl acetate and mixtures thereof; and

(B) at least 40% by weight of a polymer chosen from low molecular weight polyvinylpyrrolidone, low molecular weight hydroxypropyl methylcellulose, low molecular weight hydroxypropyl cellulose, low molecular weight methylcellulose, low molecular weight hydroxyethyl cellulose, hydroxyethyl methylcellulose, maltodextrin, poloxamers, polyethylene glycols having a molecular weight strictly comprised between 3,000 and 20,000 g/mol, polyvinyl alcohols, vinyl pyrrolidone-vinyl acetate copolymers, xanthan gum, acacia gum, carrageenan gum, guar gum, carob gum, agar-agar, copolymers of methylvinyl ether and maleic anhydride or maleic acid, aminoalkyl methacrylate copolymers, in particular copolymers of butyl methacrylate, 2-dimethylaminoethyl methacrylate and methyl methacrylate 1/2/1, the polyvinyl acetate diethyl aminoacetates, the polyvinyl aminoacetals, and mixtures thereof;

the weight ratio of polymer (B)/polymer (A) being comprised between 85/15 and 50/50;

and said coating layer representing at least 30% by weight of the total weight of said coated particles,

According to a particular embodiment, said core is coated with at least one coating layer comprising:

(A) at least 25% by weight of a polymer chosen from ethylcellulose, cellulose acetate, cellulose acetate butyrate, ammonio (meth)acrylate copolymers, polymers and copolymers of (meth)acrylic acid esters, and mixtures thereof; and

(B) at least 40% by weight of a polymer chosen from low molecular weight polyvinylpyrrolidone, low molecular weight hydroxypropyl methylcellulose, low molecular weight hydroxypropyl cellulose, low molecular weight methylcellulose, low molecular weight hydroxyethyl cellulose, hydroxyethyl methylcellulose, maltodextrin, poloxamers, polyethylene glycols having a molecular weight strictly comprised between 3,000 and 20,000 g/mol, polyvinyl alcohols, vinyl pyrrolidone-vinyl acetate copolymers, xanthan gum, acacia gum, carrageenan gum, guar gum, carob gum, agar-agar, copolymers of methylvinyl ether and maleic anhydride or maleic acid, aminoalkyl methacrylate copolymers, in particular copolymers of butyl methacrylate, 2-dimethylaminoethyl methacrylate and methyl methacrylate 1/2/1, the polyvinyl acetate diethyl aminoacetates, the polyvinyl aminoacetals, and mixtures thereof:

the weight ratio of polymer (B)/polymer (A) being comprised between 75/25 and 50/50,

Within the meaning of the invention, the term “ethylcellulose” refers to any of the ethylcelluloses.

Within the meaning of the invention, the expression “cellulose acetate” refers to any of the cellulose acetates.

Within the meaning of the invention, the expression “cellulose acetate butyrate” refers to any of the cellulose acetates butyrates.

Within the meaning of the invention, the expression “aminoalkyl methacrylate copolymers” and the expression “amino methacrylate copolymer” will be used interchangeably.

Throughout the description, the expressions “polymers and copolymers of (meth)acrylic acid esters” and “ethyl acrylate and methyl methacrylate copolymers” will further be used interchangeably.

According to another of its aspects, it also relates to the use of coated particles consisting of a non-monocrystalline core containing at least one active compound,

said core being coated with at least one coating layer comprising:

(A) at least 15% by weight of a polymer chosen from ethylcellulose, cellulose acetate, cellulose acetate butyrate, ammonio (meth)acrylate copolymers, polymers and copolymers of (meth)acrylic acid esters, polyvinyl acetate and mixtures thereof; and

(B) at least 40% by weight of a polymer chosen from low molecular weight polyvinylpyrrolidone, low molecular weight hydroxypropyl methylcellulose, low molecular weight hydroxypropyl cellulose, low molecular weight methylcellulose, low molecular weight hydroxyethyl cellulose, hydroxyethyl methylcellulose, maltodextrin, poloxamers, polyethylene glycols having a molecular weight strictly comprised between 3,000 and 20,000 g/mol, polyvinyl alcohols, vinyl pyrrolidone-vinyl acetate copolymers, xanthan gum, acacia gum, carrageenan gum, guar gum, carob gum, agar-agar, copolymers of methylvinyl ether and maleic anhydride or maleic acid, aminoalkyl methacrylate copolymers, in particular copolymers of butyl methacrylate, 2-dimethylaminoethyl methacrylate and methyl methacrylate 1/2/1, the polyvinyl acetate diethyl aminoacetates, the polyvinyl aminoacetals, and mixtures thereof:

the weight ratio polymer (B)/polymer (A) being comprised between 85/15 and 50/50;

and said coating layer representing at least 30% by weight of the total weight of said coated particles,

for preparing an oral dosage form, in particular a multiparticulate oral dosage form, for the immediate release of said active compound.

Within the meaning of the invention, by “oral dosage form” or “multiparticulate oral dosage form”, is meant any form consisting of several particles or units containing the active compound, in contrast to monolithic or unitary forms consisting of one single unit. Within the meaning of the invention, the following terms “units” or “particles” will be used interchangeably. The particles or units contained in the oral dosage form, in particular in the multiparticulate oral dosage form, are individually coated. They can be microbeads, microspheres, pellets, particles or minitablets. The oral dosage form, in particular in the multiparticulate oral dosage form, consisting of coated particles or units can be in form of a tablet, sachet or capsule or any other suitable form.

The coated particles containing the active compound and forming the oral dosage form, in particular in the multiparticulate oral dosage form, according to the invention are advantageously resistant to crushing, so that it is very difficult to break their coating and obtain a fine powder of the active compound. After crushing the oral dosage form, it is thus very difficult to obtain the active compound in fine or finely divided powder form, i.e. in form of small size crystals or particles having an average diameter generally comprised between 5 and 50 microns. Such a crystal or particle size is known to increase the dissolution rate of the active compound, thus promoting its rapid absorption through the nasal mucous membranes but also extraction for preparing an injectable product.

These coated particles containing the active compound and forming the oral dosage form, in particular in the multiparticulate oral dosage form according to the invention are hereafter designated as “coated particles”.

The coated particles containing the active compound exhibit, after crushing, a change in average diameter of less than or equal to 20%, preferably less than or equal to 15%, preferentially less than or equal to 10% and more preferentially less than or equal to 5%. This low range of change in average diameter refers to resistance to crushing of the tested particles.

Thus, by the expression “resistant to crushing” or “crush-resistant”, is meant that the population of coated particles containing the active compound, after crushing according to the operating method described hereafter, has a size distribution, so that the difference between the two average diameters, determined by analytical sieving, before crushing (D1) and after crushing (D2) respectively, i.e. change in average diameter calculated according to the following formula:

|(D1−D2)/D1|,

is, in absolute value, less than or equal to 20%, preferably less than or equal to 15%, preferentially less than or equal to 10%, and more preferentially less than or equal to 5%.

This resistance to crushing of coated particles is assessed according to the following protocol, based on the mortar and pestle technique and implemented in the examples.

Crushing Test for Coated Particles

The crushing test is carried out by means of an automatic mortar grinder, such as RM 200 mortar grinder from Retsch equipped with a stainless steel mortar and a stainless steel pestle. The pestle is adjusted in horizontal off-centre position. The vertical position of the pestle is set to position 8. A 20 g sample of particles is introduced into the mortar and ground for 1 minute. The resulting powder is entirely collected and its average diameter is determined by analytical sieving, as detailed hereafter.

This test is representative of the crushing methods usually implemented by mis-users, such as for example: pestle and mortar, coffee mill, crushing between two spoons, crunching and chewing, etc.

The oral dosage form, in particular the multiparticulate oral dosage form, according to the invention immediately releases the active compound it contains.

Within the meaning of the invention, the expression “for immediate release”, is meant to describe the ability of the oral dosage form, in particular the multiparticulate oral dosage form, to release at least 75% of the active compound within a period of less than or equal to 45 minutes in a 0.1N hydrochloric acid solution.

The amount of active compound released is assessed by means of a dissolution Apparatus 2 (Paddle apparatus), in 900 mL of a 0.1N hydrochloric acid solution at 37° C. and at a paddle rotating speed of 100 rpm, according to the method of the European Pharmacopoeia 7th Edition 2012 (7.5), Chapter 2.9.3—Dissolution test for solid dosage forms.

Preferably, the coated particles and the oral dosage form of the invention release at least 80% of the active compound, in particular at least 90% of the active compound, within a period of less than or equal to 45 minutes, in particular less than or equal to 30 minutes.

To make reading easier, polymers chosen from the aforementioned list of polymers (A) will be referred to, hereinafter, as “water-insoluble polymers” or “polymers (A)”; and polymers chosen from the aforementioned list of polymers (8) as “polymer soluble in a 0.1N hydrochloric acid solution” or “polymers (B)”.

It is understood that, unless otherwise specified, the term “polymer” is used in the text to designate equally one single polymer or a mixture of polymers.

Similarly. “active compound” is meant to designate equally one single active compound or a mixture of active compounds.

Other characteristics, advantages and embodiments of the coated particles will become more apparent on reading the description which follows.

In the remainder of the text, the expressions “comprised between . . . and . . . ” and “varying from . . . to . . . ” are equivalent and are meant to signify that the limits are inclusive, unless otherwise specified.

Unless otherwise indicated, the expression “comprising a” must be understood as “comprising at least one” and “comprising one or more”.

Within the meaning of the invention, the term “approximately” is meant to signify that the value which follows this term is verified taking account of the limits of experimental error acceptable to a person skilled in the art.

Coated Particles

The coated particles according to the invention comprise a composition and a structure which are adjusted, on the one hand, to render them resistant to crushing and, on the other hand, to obtain immediate release of the active compound they contain.

The coated particles according to the invention are structurally organized in a core containing the active compound and coated or film-coated with a coating.

According to a particular embodiment, the coated particles according to the invention have an average diameter less than or equal to 1,000 μm.

Preferably, the coated particles have an average diameter comprised between 50 and 600 μm, in particular between 100 and 400 μm, more particularly between 150 and 300 μm.

The average diameter of the coated particles is determined by analytical sieving, in particular using a sieve tower containing a sieve base and different sieves with decreasing mesh openings, as described more precisely in the examples which follow. The sieve tower comprises more particularly the sieves having the following mesh openings: 1,000, 710, 500, 250, 100 and 50 μm.

The average diameter of the coated particles by analytical sieving is calculated according to the following formula:

$$D = \frac{{\sum }_{i = 1}^{n} {m}_{i} {d}_{i}}{{\sum }_{i = 1}^{n} {m}_{i}}$$

with:
i: fraction of product comprised between the sieves with mesh openings dᵢₘₐₓ and dᵢₘᵢₙ
mᵢ: weight of product of the fraction i
dᵢ: average diameter of the fraction i, calculated according to:

$$di = \frac{{d}_{ima x} + {d}_{imi n}}{2}$$

Core of the Coated Particles

According to one aspect of the invention, the core of the coated particles contains one or more active compounds. The core of the coated particles is not monocrystalline.

By the expression “non-monocrystalline” is meant to exclude, within the meaning of the invention, cores formed of one unique crystal of active compound.

Active Compounds

The coated particles according to the invention are compatible with a great variety of active compounds and are not limited to the implementation of the active compounds more particularly described hereafter. According to the examples below, the coated particles according to the invention enable immediate release of the active compound contained therein, remaining resistant to crushing thanks to their structural organization and their coating composition. These properties are verified for active compounds having different natures, as also demonstrated in the examples.

The coated particles according to the invention are particularly advantageous for active compounds, in particular pharmaceutical or veterinary, the abuse of which can give rise to addictive behaviour, such as for example the active compounds classified as psychotropic medications or narcotics.

Thus, the active compound contained in the coated particles according to the invention can be, for example, chosen from one of the following families of active substances: amphetamines, anorexigens, antidepressants, antiepileptics, antiparkinsonians, anxiolytics, barbiturates, benzodiazepines, hypnotics, narcotics, neuroleptics, opioids, psychostimulants and psychotropics.

Thus, in a preferred embodiment, the active compound is chosen from psychotropics and narcotics, preferably chosen from oxybate, its pharmaceutically acceptable salts, polymorphs and solvates, and opioids and opioid analogues which are more preferably chosen from oxycodone, oxymorphone, hydromorphone, hydrocodone, tramadol, morphine, buprenorphine, dextropropoxyphene, propoxyphene, codeine, fentanyl, alfentanyl, remifentanyl, methadone, pethydine, nalbuphine, levomethadyl acetate, difenoxine, diphenoxylate, loperamide, pentazocine, butorphanol, levorphanol, tapentadol and their pharmaceutically acceptable salts, polymorphs and solvates, more particularly chosen from oxycodone hydrochloride, hydromorphone hydrochloride, oxymorphone hydrochloride or morphine sulphate, and their pharmaceutically acceptable salts, polymorphs and solvates.

According to a particular embodiment, the active compound is an opioid or an opioid analog.

More precisely, the active compound utilized can be chosen from oxycodone, oxymorphone, hydromorphone, hydrocodone, tramadol, morphine, buprenorphine, dextropropoxyphene, propoxyphene, codeine, fentanyl, alfentanyl, remifentanyl, methadone, pethydine, nalbuphine, levomethadyl acetate, difenoxine, diphenoxylate, loperamide, pentazocine, butorphanol, levorphanol, tapentadol and their pharmaceutically acceptable salts, polymorphs and solvates.

It can for example be oxycodone hydrochloride, hydromorphone hydrochloride, oxymorphone hydrochloride or morphine sulphate.

According to another embodiment, the active compound is oxybate or its pharmaceutically acceptable salts, polymorphs and solvates.

According to a particularly preferred embodiment, the core of the coated particles according to the invention is in compact and overall spherical form.

According to a particular embodiment, the core of the coated particles according to the invention has an average diameter of less than or equal to 450 μm, preferably less than or equal to 300 μm, preferentially less than or equal to 250 μm, in particular comprised between 80 and 250 μm.

The core of the coated particles can be:
a non-monocrystalline granule containing only the active compound;
a granule containing the active compound optionally mixed with at least one excipient, such as for example a binding agent, a diluent or a filler, a surfactant, a disintegrant, a buffering agent, an anti-foaming agent;
a granule consisting of a carrier particle, further called inert core, covered with a layer comprising the active compound optionally mixed with at least one excipient, such as for example a binding agent, a diluent or a filler, a surfactant, a disintegrant, a buffering agent, an anti-foaming agent.

The choice and adjustment of the amounts of these excipients are clearly within the expertise of a person skilled in the art.

The core of the coated particles according to the invention can thus comprise, in addition to the active compound, in particular as described previously, at least one binding agent, in particular selected from:
low molecular weight hydroxypropyl cellulose (such as for example Klucel® EF from Aqualon-Hercules), low molecular weight hydroxypropyl methylcellulose (or hypromellose) (such as for example Methocel® E3 or E5 from Dow), low molecular weight methylcellulose (such as for example Methocel® A15 from Dow);
low molecular weight polyvinyl pyrrolidone (or povidone) (such as for example Plasdone® K29/32 from ISP or Kollidon® 30 from BASF), vinyl pyrrolidone and vinyl acetate copolymer (or copovidone) (such as for example Plasdone®: S630 from ISP or Kollidon® VA 64 from BASF);
dextrose, pregelatinized starches, maltodextrin;

and mixtures thereof.

Low molecular weight hydroxypropyl cellulose corresponds to grades of hydroxypropyl cellulose having a molecular weight of less than 800,000 g/mol, preferably less than or equal to 400,000 g/mol, and in particular less than or equal to 100,000 g/mol. Low molecular weight hydroxypropyl methylcellulose (or hypromellose) corresponds to grades of hydroxypropyl methylcellulose the solution viscosity of which, for a 2% solution in water and at 20° C., is less than or equal to 1,000 mPa·s, preferably less than or equal to 100 mPa·s and in particular less than or equal to 15 mPa·s. Low molecular weight polyvinyl pyrrolidone (or povidone) corresponds to grades of polyvinyl pyrrolidone having a molecular weight of less than or equal to 1,000,000 g/mol, preferably less than or equal to 800,000 g/mol, and in particular less than or equal to 100,000 g/mol.

Preferably, the binding agent is chosen from low molecular weight polyvinylpyrrolidone (also called povidone; for example, Plasdone® K29/32 from ISP), low molecular weight hydroxypropylcellulose (for example, Klucel® EF from Aqualon-Hercules), low molecular weight hydroxypropyl methylcellulose (also called hypromellose; for example, Methocel® E3 or E5 from Dow) and mixtures thereof.

The surfactant optionally present, as described previously, in the core of the particles according to the invention can be chosen from phospholipids, polysorbates, polyoxyethylene stearates, fatty acid esters derived from polyoxyethylenated sorbitol, polyoxyethylenated hydrogenated castor oils, polyoxyethylenated alkyl ethers, glycerol monooleate, and mixtures thereof.

The diluent or filler optionally present, as described previously, in the core of the particles according to the invention, can be chosen from lactoses, saccharoses, mannitol (for example Pearlitol® grades from Roquette and in particular Pearlitol® SD200), xylitol, erythritol, sorbitols, microcrystalline cellulose (for example Avicel® products from FMC Biopolymer), calcium carbonates (for example Omyapure 35 from Omya), di- and tricalcium phosphates (for example Dicafos® and Tricafos® from Budenheim), magnesium oxide, talc, magnesium silicate and mixtures thereof.

The disintegrant optionally present, as described previously, in the core of the particles according to the invention, can be chosen from starches and pregelatinized starches, carboxymethyl cellulose, croscarmellose, crospovidone (for example Polyplasdone® grades from ISP, Kollidon® CL from BASF), low substituted hydroxypropyl cellulose, and mixtures thereof.

The buffering agent optionally present, as described previously, in the core of the particles according to the invention, can be chosen from citric acid, tartaric acid, adipic acid, boric acid, malic acid, maleic acid, phosphoric acid, glycine, methionine, sodium bicarbonate, calcium carbonate, calcium phosphate, sodium phosphate, potassium phosphate, ethanolamine, sodium glutamate, sodium citrate, potassium citrate, sodium acetate, sodium borate, sodium hydroxide, mixtures thereof, or any other buffering agent known in the art.

The anti-foaming agent optionally present, as described previously, in the core of the particles according to the invention, can be chosen from simethicone, dimethicone.

According to a particular embodiment, the core of the coated particles is formed by a carrier particle, or inert core, covered with a layer comprising at least said active compound(s). Said carrier particles can be:
crystals or spheres of lactose, sucrose (such as for example Compressue® PS from Tereos), microcrystalline cellulose (such as for example Avicel® from FMC Biopolymer, Cellet® from Pharmatrans or Celphere® from Asahi Kasci), sodium chloride, calcium carbonate (such as for example Omyapure® 35 from Omya), sodium hydrogen carbonate, dicalcium phosphate (such as for example Dicafos® AC 92-12 from Budenheim) or tricalcium phosphate (such as for example Tricafos® SC93-15 from Budenheim);
composite spheres or granules, for example sugar spheres comprising sucrose and starch (such as for example Suglets® from NP Pharm), spheres of calcium carbonate and starch (such as for example Destab® 90 S Ultra 250 from Particle Dynamics) or spheres of calcium carbonate and maltodextrin (Hubercal® CCG4100 from Huber).

The carrier particles can also be any other particles of pharmaceutically acceptable excipient(s) such as for example particles of hydroxypropyl cellulose (such as for example Klucel® from Aqualon Hercules), guar gum particles (such as for example Grinsted® Guar from Danisco), xanthan particles (such as for example Xantural® 180 from CPKelco).

According to a particular embodiment of the invention, the carrier particles are sugar spheres or microcrystalline cellulose spheres, such as for example Cellet® 90, Cellets® 100 or Cellets® 127 marketed by Pharmatrans or any sugar or microcrystalline cellulose spheres having a volume mean diameters equal to approximately 95 μm, 170 μm and 140 μm, or also Celphere® CP 203, Celphere® SCP 100 and more particularly the fraction of Celphere® SCP 100 less than 100 μm or any microcrystalline cellulose spheres with the volume mean diameter of approximately 100 μm, commercialized by Asai Kasei, or also dicalcium phosphate particles, for example Dicafos® AC 92-12 and more particularly the fraction of Dicafos® AC 92-12 comprised between 50 and 100 μm or any dicalcium phosphate particles having a volume mean diameter of approximately 75 μm.

According to a particularly preferred embodiment, the active layer covering the carrier particle for forming the core of the coated particles of the invention comprises, in addition to the active compound, at least one binding agent.

The layer containing at least said active compound and covering the carrier particle, or inert core, can represent at least 10% by weight, preferably 20% by weight, preferably at least 30% by weight, preferably at least 50% by weight, preferably at least 60% by weight, more preferably from 70 to 95% by weight and in particular from 80 to 90% by weight of the total weight of the core of the coated particle.

Coating of the Particles

Within the framework of the present invention, the core of the coated particles, containing the active compound, is covered with a coating the composition and thickness of which are precisely adjusted in order, on the one hand, to provide the immediate release of said active compound and, on the other hand, to contribute to impart crush-resistance to the coated particles according to the crushing test described previously.

According to a first of its aspects, the coating layer, also called coating or film-coating, which covers the core of the coated particles represents at least 30% by weight of the total weight of the coated particles; in other words, the coated particles have an average mass coating rate of at least 30%.

More particularly, the coating can represent from 30 to 60% by weight, in particular from 30 to 55% by weight and more particularly from 30 to 50% by weight of the total weight of the coated particles.

According to another of its aspects, the coating layer comprises:
at least 15% by weight of water-insoluble polymer, and
at least 40% by weight of polymer soluble in a 0.1N hydrochloric acid solution.

According to a particular embodiment, the coating layer comprises:
at least 25% by weight of water-insoluble polymer, and
at least 40% by weight of polymer soluble in a 0.1N hydrochloric acid solution.

The weight ratio between the polymer soluble in a 0.1N hydrochloric acid solution and the water-insoluble polymer is comprised between 85/15 and 50/50, preferably between 75/25 and 50/50, preferably between 70/30 and 50/50, and preferentially between 60/40 and 50/50.

The water-insoluble polymer is chosen from:
ethylcellulose,
cellulose acetate,
cellulose acetate butyrate.
ammonio (meth)acrylate copolymers,
polymers and copolymers of (meth)acrylic acid esters,
polyvinyl acetate,
and mixtures thereof.

Preferably, the water-insoluble polymer is chosen from ethylcellulose, cellulose acetate and ammonio (meth)acrylate copolymers.

As examples of water-insoluble polymers which can be used according to the invention, there may be mentioned: ethylcellulose, in particular marketed under the name Ethocel® by Colorcon, and more particularly the Ethocel® 20 grade; cellulose acetate, in particular marketed under the name CA 398-10NF by Eastman; cellulose acetate butyrate, in particular marketed under the name CAB 171-15 by Eastman; ammonio (meth)acrylate copolymers, in particular marketed under the names Eudragit® RL and Eudragit® RS by Evonik; polymers and copolymers of (meth)acrylic acid esters, in particular marketed under the names Eudragit® NE and Eudragit® NM.

According to a particular embodiment, the water-insoluble polymer is present in a content comprised between 15 and 60% by weight, preferably between 25 and 50% by weight, in particular between 25 and 45% by weight, relative to the total weight of said coating layer.

In particular, the coating layer can comprise from 30 to 45% by weight of water-insoluble polymer.

The polymer soluble in a 0.1N hydrochloric acid solution is chosen from:
low molecular weight polyvinyl pyrrolidone (PVP);
low molecular weight hydroxypropyl methylcellulose (or hypromellose or HPMC), low molecular weight hydroxypropyl cellulose (HPC), low molecular weight methylcellulose, low molecular weight hydroxyethyl cellulose, hydroxyethyl methylcellulose;
mahodextrin;
poloxamers which are ethylene oxide, propylene oxide and ethylene oxide triblock polymers;
polyethylene glycols having a molecular weight strictly comprised between 3,000 and 20,000 g/mol;
polyvinyl alcohols;
vinyl pyrrolidone-vinyl acetate copolymers;
xanthan gum;
acacia gum;
carrageenan gum;
guar gum;
carob gum;
agar-agar;
copolymers of methylvinyl ether and maleic anhydride or maleic acid;
aminoalkyl methacrylate copolymers, in particular copolymers of butyl methacrylate, 2-dimethylaminoethyl methacrylate and methyl methacrylate 1/2/1;
polyvinyl acetate diethyl aminoacetate;
polyvinyl aminoacetals;
and mixtures thereof.

Low molecular weight polyvinyl pyrrolidone (or povidone) corresponds to grades of polyvinyl pyrrolidone having a molecular weight of less than or equal to 1,000,000 g/mol, preferably less than or equal to 800,000 g/mol, and in particular less than or equal to 100,000 g/mol.

Low molecular weight hydroxypropyl methylcellulose (or hypromellose) corresponds to grades of hydroxypropyl methylcellulose the solution viscosity of which, for a 2% solution in water at 20° C., is less than 1,000 mPa·s, preferably less than or equal to 100 mPa·s and in particular less than or equal to 15 mPa·s.

Low molecular weight hydroxypropyl cellulose corresponds to grades of hydroxypropyl cellulose having a molecular weight of less than 800,000 g/mol, preferably less than or equal to 400,000 g/mol, and in particular less than or equal to 100,000 g/mol.

Low molecular weight methylcellulose corresponds to grades of methylcellulose the solution viscosity of which, for a 2% solution in water at 20° C., is less than 1,000 mPa·s, preferably less than or equal to 15 mPa·s.

Low molecular weight hydroxyethyl cellulose corresponds to grades of hydroxyethyl cellulose the solution viscosity of which, for a 2% solution in water at 25SC, having a viscosity in solution at 2% in water at 25° C. is less than 1,000 mPa·s.

According to a particularly preferred embodiment, the polymer soluble in a 0.1N hydrochloric acid solution is chosen from low molecular weight polyvinyl pyrrolidone, low molecular weight hydroxypropyl methylcellulose, low molecular weight hydroxypropyl cellulose and copolymers of butyl methacrylate, 2-dimethyl aminoethyl methacrylate and methyl methacrylate 1/2/1.

As examples of polymers soluble in a 0.1N hydrochloric acid solution which can be used according to the invention, there may be mentioned copolymers of butyl methacrylate, 2-dimethylaminoethyl methacrylate and methyl methacrylate 1/2/1, for example marketed under the name Eudragit® E by Evonik, and in particular Eudragit® E100 and Eudragit® EPO grades; polyvinyl pyrrolidone, also called povidone, with a low molecular weight, for example marketed under the trade name Plasdone® by ISP, more particularly the Plasdone® K29/32 grade; hydroxypropyl methylcellulose also called hypromellose, with a low molecular weight, for example marketed under the trade name Methocel® by Colorcon and more particularly the Methocel® E3 grade; polyvinyl acetate diethyl aminoacetate, for example marketed under the name AEA® by Sankyo Company Limited (JP).

According to a preferred embodiment, the polymer soluble in a 0.1N hydrochloric acid solution as defined previously is present in an amount comprised between 40 to 85% by weight, preferably between 40 and 75% by weight, and still more preferably from 45 to 60% by weight relative to the total weight of said coating layer.

Plasticizer

The coating of the coated particles according to the invention can also comprise at least one plasticizer.

By “plasticizer” is meant equally one single plasticizer or a mixture of plasticizers.

One skilled in the art is able to choose the suitable plasticizer.

The plasticizer can in particular be chosen from:
glycerol and its esters, and preferably from the acetylated glycerides, glycerol monostearate, glyceryl triacetate, glyceryl tributyrate.
phthalates, and preferably from dibutyl phthalate, diethyl phthalate, dimethyl phthalate, dioctyl phthalate,
citrates, and preferably from acetyl tributyl citrate, acetyl triethyl citrate, tributyl citrate, triethyl citrate,
sebacates, and preferably from diethyl sebacate, dibutyl sebacate,
adipates,
azelates,
benzoates,
chlorobutanol,
polyethylene glycols having a molecular weight of less than or equal to 3,000 g/mol;
vegetable oils,
the fumarates, preferably diethyl fumarate,
the malates, preferably diethyl malate,
the oxalates, preferably diethyl oxalate,
the succinates; preferably dibutyl succinate.
the butyrates,
the cetyl alcohol esters,
the malonates, preferably diethyl malonate,
castor oil,
and mixtures thereof.

The plasticizer is more particularly chosen from triethyl citrate and polyethylene glycols having a molecular weight of less than or equal to 3,000 g/mol.

In particular, the plasticizer is present in an amount of less than or equal to 30% by weight, preferably less than or equal to 20% by weight, preferably less than or equal to 15% by weight, and, more preferably from 5% to 15% by weight, relative to the total weight of said coating layer.

According to a particularly preferred embodiment, the polymer soluble in a 0.1N hydrochloric acid solution according to the invention, the water-insoluble polymer and the plasticizer represent at least 70% by weight, in particular at least 80% by weight and more particularly at least 90% by weight of the total weight of the coating layer.

As a non-limiting illustration of the coated particles according to the invention, there can in particular be mentioned a composition the coating of which represents between 30 and 55% by weight relative to the total weight of coated particles and comprises:
30 to 45% by weight water-insoluble polymer chosen from ethylcellulose or cellulose acetate;
45 to 60% by weight polymer soluble in a 0.1N hydrochloric acid solution chosen from copolymers of butyl methacrylate, 2-dimethylaminoethyl methacrylate and methyl methacrylate 1/2/1; low molecular weight polyvinyl pyrrolidone and low molecular weight hydroxypropyl methylcellulose; and
0 to 15% by weight plasticizer chosen from triethyl citrate and polyethylene glycol having a molecular weight of approximately 400 g/mol.

According to a particular embodiment, the coating of the coated particles represents between 40 and 55% by weight relative to the total weight of coated particles and comprises:
30 to 45% by weight ethylcellulose;
45 to 60% by weight copolymers of butyl methacrylate, 2-dimethylaminoethyl methacrylate and methyl methacrylate 1/2/1; and
0 to 10% by weight triethyl citrate or polyethylene glycol having a molecular weight of approximately 400 g/mol.

According to a particular embodiment, the coating of the coated particles represents between 40 and 55% by weight relative to the total weight of coated particles and comprises:
30 to 45% by weight ethylcellulose;
45 to 60% by weight low molecular weight polyvinyl pyrrolidone; and
0 to 10% by weight triethyl citrate or polyethylene glycol having a molecular weight of approximately 400 g/mol.

According to a particular embodiment, the coating comprises at most 30% by weight filler, in particular less than 20% by weight, preferably less than 10% by weight of filler, relative to the total weight of said coating, or is even completely free of filler.

In particular, the filler can be talc.

Of course, the coating can comprise various other additional additives conventionally used in the field of coating. These can be, for example:
pigments and colorants, such as for example titanium dioxide, calcium sulphate, calcium carbonate, iron oxides, natural or synthetic food colouring agents;
anti-foaming agents, such as for example simethicone, dimethicone;
surfactants, such as for example phospholipids, polysorbates, polyoxyethylene stearates, esters of fatty acid and polyoxyethylenated sorbitol, polyoxyethylenated hydrogenated castor oils, polyoxyethylenated alkyl ethers, glycerol monooleate,
and mixtures thereof.

According to a particular embodiment of the invention, the coating of the particles according to the invention contains no active compound.

According to one embodiment, the coating layer does not comprise any compounds other than the abovementioned polymers, and the optional plasticizer(s).

The coating of the coated particles according to the invention can comprise a single coating layer or several coating layer formed in successive steps. According to a particularly preferred embodiment variant, it is composed of a single coating layer as described previously.

Particles, for which the structure and coating composition do not comply with the invention, do not resist crushing at the required scale according to the previously presented invention and/or do not enable immediate release of the active compound contained in said coated particles and/or are not prepared according a cheap and rapid process.

Preparation of the Coated Particles

Core of the Coated Particles

The core of the coated particles of the invention can be obtained according to several techniques such as for example:
agglomeration of the active compound sprayed preferably in the molten state, such as for example Glatt ProCell™ technique, or;
extrusion and spheronization of the active compound, optionally with one or more physiologically acceptable excipient(s), or,
wet granulation of the active compound, optionally with one or more physiologically acceptable excipient(s), or;
compacting of the active compound with optionally one or more physiologically acceptable excipient(s), or;
granulation and spheronization of the active compound with optionally one or more physiologically acceptable excipient(s), the spheronization being carried out for example in a fluidized bed apparatus equipped with a rotor, in particular according the Glatt CPS™ technique or;
spraying of the active compound with optionally one or more physiologically acceptable excipient(s), for example in a fluidized bed type apparatus equipped with zig-zag filter, in particular according the Glatt MicroPx™ technique;
spraying, for example in a fluidized bed apparatus optionally equipped with a Würster tube, of the active compound, with optionally one or more physiologically acceptable excipient(s), in dispersion or in solution in an aqueous or organic solvent on a carrier particle.

Coated Particles

According to a preferred embodiment, the coating layer of the coated particles is obtained by spraying, in particular in a fluidized bed apparatus, a solution, suspension or dispersion comprising at least one water-insoluble polymer (A) as defined previously, at least one polymer (B) soluble in a 0.1N hydrochloric acid solution as defined previously, and optionally at least one plasticizer, onto said cores comprising the active compound, in particular the cores as previously described and obtained by application of a layer containing at least said active compound onto the surface of a carrier particle or inert core.

Preferably, the coating is formed by spraying in a fluidized bed apparatus equipped with a Würster and according to an upward spray orientation (bottom spray).

Said coating solution, suspension or dispersion comprises water, one or more organic solvent(s), or mixtures thereof.

The organic solvent is chosen from the solvents known to a person skilled in the art. As examples, there can be mentioned acetone, isopropanol, ethanol and mixtures thereof.

According to a preferred embodiment, the solvent of the coating solution, suspension or dispersion comprises less than 40% by weight, in particular less than 30% by weight, in particular less than 20% by weight, preferably less than 10% by weight water, relative to the total weight of said solvent.

For example, it can be an acetone/isopropanol mixture (60/40 w/w), an acetone/water mixture (90/10 w/w), or an ethanol/water mixture (70/30 w/w).

Preferentially, the polymers and, if appropriate, the plasticizer and filler are sprayed in solute state i.e. in solubilized form in a solvent in order to promote the homogeneity of the formed coating.

According to a particularly preferred embodiment, the solution, suspension or dispersion is free of filler.

Use of the Coated Particles

As specified previously, the present invention also relates to the use of such coated particles for the preparation of an oral dosage form for the immediate release of an active compound.

The use of such coated particles is particularly advantageous for the active compounds which may be diverted from their normal use, in particular psychotropic medications and narcotics, and more particularly chosen from the active compounds described previously. In particular, the active compound is chosen from opioids and more particularly from oxycodone, oxymorphone, hydromorphone, hydrocodone, tramadol, morphine and their pharmaceutically acceptable salts or hydrates.

Oral Dosage Forms, in Particular Multiparticulate Oral Dosage Forms

Viscosity Modifying Agent

According to a particular embodiment, the oral dosage form, in particular the multiparticulate oral dosage form, according to the invention can comprise, in addition to the coated particles for the immediate release of active compound according to the invention, at least one viscosity modifying agent.

More particularly, the viscosity modifying agent has the purpose, when the intact or crushed oral dosage form, in particular multiparticulate oral dosage form, is introduced into a small volume of injectable solvent, in particular into a volume less than or equal to 10 ml, of converting the corresponding mixture into a non-homogeneous paste, which is too viscous to be filtered or injected through a needle of 25 gauge or higher, e.g. 25, 26, 27, 29, 30, or 31 gauge, thus preventing obtaining an injectable liquid containing the active compound in an immediately available form.

An oral dosage form, in particular the multiparticulate oral dosage form, according to the invention can thus comprise at least one viscosity modifying agent distinct from the coated particles comprising the active compound.

Preferably, the viscosity modifying agent comprised into the oral dosage form, in particular into the multiparticulate oral dosage, form according to the invention is entirely distinct from the coated particles comprising the active compound.

According to a preferred embodiment, the viscosity modifying agent is chosen from viscosity modifying agents which are soluble in at least one of the solvents chosen from water, alcohols, ketones and mixtures thereof.

According to a preferred embodiment, the viscosity modifying agent is capable of increasing the viscosity of a small volume (between 2.5 mL and 10 mL) of solvent, in order to prevent injection, in particular by intra-venous route. In fact, the viscosity becomes so high that the drawing off of the mixture formed by the introduction of the solid oral dosage form according to the invention in a small volume of injectable solvent by a syringe becomes impossible.

According to a particular embodiment, an oral dosage form according to the invention can advantageously comprise a mixture of several viscosity modifying agents which will be effective both in the case of extraction in aqueous phase and in an organic solvent.

According to a particular embodiment, the viscosity modifying agent contributes to prevent misuse of the oral dosage form by inhalation since it is able to form a gel in contact with nasal mucous membranes, wherein said gel will hamper the diffusion of the active compound towards the mucous membranes and thus its absorption.

As regards the amount of viscosity modifying agent, it can easily be determined by a person skilled in the art. This amount advantageously corresponds to the minimum amount necessary to bring the viscosity of 2.5 mL of extraction liquid to a value greater than or equal to 100 mPa·s, preferably 200 mPa·s, and still more preferably above 500 mPa·s, and still better 1,000 mPa·s.

According to one embodiment, the oral dosage form according to the invention comprises up to 500 mg viscosity modifying agent. In particular, the oral dosage form according to the invention comprises between 5 and 500 mg, preferably between 10 and 250 mg, preferably between 10 and 100 mg, more preferentially between 10 and 80 mg and in particular between 15 and 60 mg viscosity modifying agent.

According to a particular embodiment, the viscosity modifying agent is chosen from:
polyacrylic acids, in particular carbomers, for example Carbopol®,
polyalkylene glycols, for example polyethylene glycols having a molecular weight of greater than or equal to 20,000 g/mol,
polyalkylene oxides, for example the polyethylene oxides or polyoxyethylene,
high molecular weight polyvinyl pyrrolidones,
gelatins,
polysaccharides, preferably chosen from sodium alginate, pectins, guar gum, xanthans, carrageenans, gellans, high molecular weight hydroxypropyl cellulose, high molecular weight hydroxypropyl methylcellulose, high molecular weight methylcellulose, high molecular weight hydroxyethyl cellulose and carboxymethyl cellulose,
and mixtures thereof.

High molecular weight polyvinyl pyrrolidone corresponds to grades of polyvinyl pyrrolidone having a molecular weight of greater than 1,000,000 g/mol.

High molecular weight hydroxypropyl cellulose corresponds to grades of hydroxypropyl cellulose having a molecular weight of greater than or equal to 800,000 g/mol, and preferably greater than or equal to 1,000,000 g/mol.

High molecular weight hydroxypropyl methylcellulose corresponds to grades of hydroxypropyl methylcellulose the solution viscosity of which, for a 2% solution in water at 20° C., is greater than or equal to 1,000 mPa·s, preferably greater than or equal to 15,000 mPa·s and in particular less than or equal to 100,000 mPa·s.

High molecular weight methylcellulose corresponds to grades of methylcellulose the solution viscosity of which, for a 2% solution in water at 20° C. is greater than or equal to 1,000 mPa·s.

High molecular weight hydroxyethyl cellulose corresponds to grades of hydroxyethyl cellulose the solution viscosity of which, for a 2% solution in water at 25° C., is greater than or equal to 1,000 mPa·s.

According to a particularly preferred embodiment, the viscosity modifying agent is a polyoxyethylene, in particular a high molecular weight polyoxyethylene, and more particularly a polyoxyethylene having an average molecular weight comprised between 1 million g/mol and approximately 8 million g/mol.

As a viscosity modifying agent, there can in particular be mentioned polyoxyethylene marketed by Dow under the reference Sentry Polyox WSR® 303.

According to a particularly preferred embodiment, the oral dosage form according to the invention, the oral dosage form according to the invention comprises between 5 and 500 mg, preferably between 10 and 250 mg, preferably between 10 and 100 mg, more preferentially between 10 and 80 mg and in particular between 15 and 60 mg polyoxyethylene, in particular high molecular weight polyoxyethylene.

The viscosity modifying agent, for example high molecular weight polyoxethylene, is in the form of particles, distinct from the coated particles for the immediate release of active compound according to the invention as described previously.

According to another embodiment, the viscosity modifying agent particles have a size distribution similar to that of the coated particles for the immediate release of active compound according to the invention, so that they cannot be separated by sieving from the coated particles comprising the active compound.

According to another embodiment, the volume mean diameter of the viscosity modifying agent particles is comprised between 0.5 and two times, preferably comprised between 0.7 and 1.5 times, still more preferably comprised between 0.8 and 1.25 times the volume mean diameter of the coated particles for the immediate release of active compound.

Presentation of the Oral Dosage Form

According to a particularly preferred embodiment, an oral dosage form according to the invention is a solid oral dosage form of tablet, capsule or sachet type.

According to a particular embodiment, the oral dosage form containing the coated particles for the immediate release of active compound also comprises one or more physiologically acceptable excipients, commonly used for formulating tablets, capsules or sachets.

According to a first embodiment variant, a solid oral dosage form of capsule or sachet type can contain, in addition to the coated particles for the immediate release of active compound:
diluents such as lactose, sucrose, sugar spheres (for example Suglets from NP Pharm), microcrystalline cellulose (for example Avicel® from FMC Biopolymer or also Cellet® from Pharmatrans or Celphere® from Asai Kasei), calcium carbonates (for example Omyapure® 35 from Omya, Destab® 90 S Ultra 250 from Particle Dynamics or Hubercal® CCG4100 from Huber), di- and tricalcium phosphates (for example Dicafos® and Tricafos® from Budenheim), magnesium oxide, calcium phosphate and sulphate;
lubricants or glidants such as stearates, in particular magnesium stearate, calcium stearate or zinc stearate, stearic acid, glycerol behenate, sodium stearyl fumarate, talc, colloidal silica;
disintegrant agents, such as starches and pregelatinized starches (for example maize starch), carboxymethyl cellulose, croscarmellose, crospovidone (for example the Polyplasdone® grades from ISP, Kollidon® CL from BASF), low substituted hydroxypropyl cellulose;
colorants or pigments, such as titanium dioxide, calcium sulphate, precipitated calcium carbonate, iron oxides, natural food colorants such as caramels, carotenoids, carmine, chlorophyllins, Rocou (or annatto), xanthophylls, anthocyans, betanin, aluminium, and synthetic food colorants
flavourings, for example strawberry, orange, banana, mint flavourings;
preservatives, such as parabens, in particular methylparaben, ethylparaben, propylparaben and butylparaben, benzoic acid and its salts (for example sodium benzoate), chlorocresol, sorbic acid and its salts, glycerine;
and mixtures thereof.

The choice of these excipients, for a solid oral dosage form of capsule or sachet type is clearly within the expertise of a person skilled in the art.

According to a particular embodiment, a capsule type solid dosage form according to the invention can in particular comprise at least one diluent in a content comprised between 0 and 80% by weight, in particular between 0.5 and 50% by weight, and more particularly between 1 and 30% by weight relative to the total weight of the capsule contents.

According to another particular embodiment, a capsule type solid dosage form according to the invention can comprise at least one lubricant or glidant in a content comprised between 0.1 and 5% by weight, in particular between 0.5 and 2% by weight relative to the total weight of the capsule contents.

According to a particular embodiment, a capsule type solid dosage form according to the invention comprises, in addition to the coated particles defined above, at least one diluent, in particular microcrystalline cellulose, and at least one lubricant or glidant, in particular chosen from magnesium stearate, colloidal silica, and mixtures thereof.

In particular, these different excipients are utilized in contents as defined previously.

According to another embodiment variant, a solid oral dosage form of tablet type can contain, in addition to the coated particles for the immediate release of active compound:
diluents or tableting agents, such as lactose, sucrose, mannitol (for example the Pearlitol® grades from Roquette and in particular Pearlitol® SD200), xylitol, erythritol, sorbitols, microcrystalline cellulose (for example Avicel® from FMC Biopolymer or also Cellet® from Pharmatrans or Celphere® from Asahi Kasei), calcium carbonates (for example Omyapure® 35 from Omya, Destab® 90 S Ultra 250 from Particle Dynamics or Hubercal® CCG4100 from Huber), di- and tricalcium phosphates (for example Dicafos® and Tricafos® from Budenheim), magnesium oxide;
lubricants or glidants such as stearates, in particular magnesium stearate, calcium stearate or zinc stearate, stearic acid, glycerol behenate, sodium stearyl fumarate, talc, colloidal silica;
binding agents, such as hydroxyethyl cellulose, ethylcellulose, hydroxypropyl cellulose (for example Klucel® from Aqualon-Hercules), hydroxypropyl methylcellulose (or hypromellose, for example Methocel® E or K and in particular Methocel® K15M from Dow), methylcellulose (for example Methocel® A 15 from Dow), polyvinyl pyrrolidone (or povidone, for example Plasdone® K29/32 from ISP, Kollidon® 30 from BASF), vinyl pyrrolidone and vinyl acetate copolymers (or copovidone, for example Plasdone® S630 from ISP, Kollidon® VA 64 from BASF), polyethylene oxide, the polyalkylenes such as for example polyethylene glycol, dextroses, pregelatinized starches, maltodextrins, polyvinyl alcohol, glycerol palmitostearate;
disintegrants, such as starches and pregelatinized starches (for example maize starch), carboxymethyl cellulose, croscarmellose, crospovidone (for example the Polyplasdone® grades from ISP, Kollidon® CL from BASF), low substituted hydroxypropyl cellulose;
colorants or pigments, such as titanium dioxide, calcium sulphate, precipitated calcium carbonate, iron oxides, natural food colorants such as caramels, carotenoids, carmine, chlorophyllins, Rocou (or annatto), the xanthophylls, anthocyans, betanin, aluminium, and synthetic food colorants;
flavourings, for example strawberry, orange, banana, mint flavourings;
preservatives, such as parabens, in particular methylparaben, ethylparaben, propylparaben and butylparaben, benzoic acid and its salts (for example sodium benzoate), chlorocresol, sorbic acid and its salts, glycerine;
and mixtures thereof.

The choice of these excipients for a solid oral dosage form of tablet type is clearly within the expertise of a person skilled in the art.

A tablet type solid form according to the invention can in particular comprise at least one tableting agent or diluent in a content comprised between 10 and 80% by weight, in particular between 30 and 75% by weight, and more particularly between 35 and 65% by weight relative to the total weight of the solid dosage form.

A solid dosage form according to the invention of tablet type can comprise at least one lubricant or glidant in a content comprised between 0.1 and 5% by weight, in particular between 0.5 and 2% by weight relative to the total weight of the solid dosage form.

According to another particular embodiment of the invention, the content of binding agent in a solid dosage form according to the invention of tablet type is less than or equal to 40% by weight, in particular less than or equal to 30% by weight, and more particularly comprised between 5 and 20% by weight relative to the total weight of the solid dosage form.

According to a particular embodiment, a solid dosage form according to the invention of tablet type comprises, in addition to the coated particles, at least one tableting agent or diluent, in particular chosen from microcrystalline cellulose, mannitol and mixtures thereof, and at least one lubricant or glidant, in particular chosen from magnesium stearate and colloidal silica and mixtures thereof, and optionally at least one binding agent, in particular chosen from hydroxypropyl methylcellulose and methylcellulose.

In particular, these different excipients are utilized in contents as defined previously.

Preparation of the Oral Dosage Forms

As seen previously, an oral dosage form according to the invention is preferably a tablet, a sachet or a capsule.

In the case of presentation in the form of a capsule or a sachet, the coated particles for the immediate release of active compound are mixed beforehand with excipients known to a person skilled in the art such as diluents, lubricants or glidant, etc. as described previously; the mixture is then distributed into capsules or sachets. Alternatively, a method can be implemented for sequential filling with the components one after another or partially or totally mixed with each other.

In the case of presentation in the form of a tablet, the coated particles for the immediate release of active compound are mixed beforehand with excipients known to a person skilled in the art such as lubricants or glidant, diluents or tableting agents etc. as described previously; the mixture is then compressed.

The compression can be carried out according to any conventional method and its implementation is clearly within the expertise of a person skilled in the art.

The tablets advantageously possess a significant breaking strength. For example, for a round tablet with a diameter of 12 mm, the hardness of the tablet can vary from 50 to 500 N, in particular from 60 to 200 N. This hardness can be measured according to the protocol described in the European Pharmacopoeia 7^{th} Edition 2012 (7.5), Chapter 2.9.8.: “Resistance to crushing of tablets”.

The final solid dosage form, in particular in the form of a tablet or capsule, can, if appropriate, be subjected to additional treatments, according to the techniques and formulae known to a person skilled in the art aimed, for example, at forming on their surface a particular film-coating or coating intended to provide them with additional properties or qualities (colour, appearance, etc.).

According to a particular embodiment, a solid dosage form according to the invention, in particular of tablet or capsule type, has a loading rate of coated particles for the immediate release of active compound, comprised between 5% and 95% by weight relative to its total weight, in particular between 10% and 90% by weight, and more particularly between 20 and 85% by weight.

Of course, other oral dosage form can be envisaged such as, for example, powders.

The examples which follow are presented by way of illustration and are non-limitative of the field of the invention.

### EXAMPLES

### Example 1

### Preparation of Coated Particles of Oxycodone Hydrochloride Complying with the Invention

### Preparation of the Coated Particles Core (or Granules)

1615.0 g oxycodone hydrochloride and 85.0 g polyvinylpyrrolidone (also named povidone; Plasdone® K29/32 from ISP) are introduced under stirring into a reactor containing 2052.1 g water and 1105.0 g ethanol. The solution is heated to 65° C. When the oxycodone hydrochloride crystals and the polyvinylpyrrolidone are dissolved, all of the solution is sprayed onto 300.0 g cellulose spheres (Cellet® 90 from Pharmatrans) in a GPCG1.1 fluidized bed apparatus in a bottom spray configuration. After spraying, the obtained product is sieved through 80 μm and 250 μm sieves and the fractions of product having a size less than 80 μm and greater than 250 μm are eliminated. 2052.1 g granules are then recovered.

Coating Step

300.0 g granules obtained according to the previous stage are coated at room temperature, in a GPCG1.1 fluidized bed apparatus equipped with a Würster tube, with 135.0 g copolymer of butyl methacrylate, 2-dimethylaminoethyl methacrylate and methyl methacrylate (Eudragit® E100 from Evonik), 135.0 g cellulose acetate (CA 398-10NF from Eastman) and 30.0 g triethyl citrate (Citrofol AI from Jungbunzlauer) dissolved in a mixture of 3105 g acetone and 345 g water (90/10 w/w). The spraying is performed with a spraying liquid flow rate of 18 g/min.

After spraying all of the solution, the coated particles are recovered. Their average coating rate is about 50%.

Crushing of the Coated Particles

Approximately 20 g coated particles prepared as described above are crushed for 1 minute using RM 200 mortar grinder from Retsch, according to the protocol previously described.

All of the powder is recovered and sieved through a nest of sieves having the following decreasing mesh openings: 1,000, 710, 500, 250, 100 and 50 μm and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 1 below.

TABLE 1

Amount of coated
particles (g)

| Size intervals | Intact | Crushed |
|---|---|---|
| 0-50 μm | 0.1 | 0.1 |
| 50-100 μm | 0.4 | 0.5 |
| 100-250 μm | 15.9 | 16.4 |
| 250-500 μm | 3.5 | 3.0 |
| 500-710 μm | 0 | 0 |
| 710-1000 μm | 0 | 0 |
| Average diameter | 207 | 202 |
| (μm) | | |

| | |
|---|---|
| Change in average | 2.8% |
| diameter | |

The average diameter of the crushed coated particles decreases by approximately 2.8% relative to that of the intact coated particles.

Hence, the coated particles prepared as described above resist crushing.

Dissolution Profiles of the Intact Coated Particles

The in vitro dissolution profile of the intact coated particles prepared as described above is determined by UV spectrometry in 900 ml 0.1 N HCl maintained at 37.0±0.5° C. and stirred by a paddle rotating at 100 rpm. The obtained dissolution profile for the intact coated particles is shown in Table 2 below.

TABLE 2

| | % oxycodone |
|---|---|
| Hours | dissolved |
| 0 | 0 |
| 0.5 | 93 |
| 1 | 93 |

After testing for 30 minutes, more than 90% of the oxycodone dose is dissolved. The profile therefore shows immediate release for the coated particles prepared above.

### Example 2

### Preparation of Coated Particles of Oxycodone Hydrochoride Complying with the Invention

Coating Step

300.0 g granules obtained in Example 1 are coated at room temperature, in a GPCG1.1 fluidized bed apparatus equipped with a Würster tube, with 180.6 g polyvinylpyrrolidone (also named povidone; Plasdone® K29/32 from ISP), 90.4 g ethylcellulose (Ethocel® 20 premium from Dow) and 30.0 g polyethylene glycol (Super Refined PEG 400 LQ MH from Croda) dissolved in a mixture of 2070 g acetone and 1380 g isopropanol (60/40 w/w). The spraying is performed with a spraying liquid flow rate of 17 g/min and lasts around 3 hours and 40 minutes.

After spraying all of the coating solution, the coated particles are recovered. Their average coating rate is about 50%.

Crushing of the Coated Particles

Approximately 20 g coated particles prepared as described above are crushed for 1 minute using a RM 200 mortar grinder from Retsch, according to the protocol previously described.

All of the powder is recovered and sieved through a nest of sieves having the following decreasing mesh openings: 1,000, 710, 500, 250, 100 and 50 μm and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 3 below.

TABLE 3

Amount of coated
particles (g)

| Size intervals | Intact | Crushed |
|---|---|---|
| 0-50 μm | 0 | 0 |
| 50-100 μm | 0.1 | 0 |
| 100-250 μm | 19.5 | 19.0 |
| 250-500 μm | 0.9 | 0.6 |
| 500-710 μm | 0 | 0 |
| 710-1000 μm | 0 | 0 |
| Average diameter (μm) | 183 | 182 |

| | |
|---|---|
| Change in average | 0.8% |
| diameter | |

The average diameter of the crushed coated particles decreases by approximately 0.8% relative to that of the intact particles.

Hence, the coated particles prepared above resist crushing.

Dissolution Profiles of the Intact Particles

The in vitro dissolution profile of the intact coated particles prepared as described above is determined by UV spectrometry in 900 ml 0.1 N HCl maintained at 37.0±0.5° C. and stirred by a paddle rotating at 100 rpm. The obtained dissolution profile is presented in Table 4 below.

TABLE 4

| | % oxycodone |
|---|---|
| Hours | dissolved |
| 0 | 0 |
| 0.25 | 100 |
| 0.5 | 100 |
| 0.75 | 100 |
| 1 | 100 |

After testing for 15 minutes, the entire dose of oxycodone is dissolved.

The profile therefore exhibits immediate release for the coated particles prepared as described above.

### Example 3

### Preparation of Coated Particles of Oxycodone Hydrochloride Complying with the Invention

Coating Step

300.0 g granules obtained in Example 1 are coated at room temperature, in a GPCG1.1 fluidized bed apparatus equipped with a Würster tube, with 135.0 g hydroxypropyl methylcellulose (also named hypromellose; Methocel® E3 from Colorcon), 135.0 g ethylcellulose (Ethocel® 20 premium from Dow) and 30.0 g polyethylene glycol (Super Refined PEG 400) LQ MH from Croda) dissolved in a mixture of 3290 g ethanol and 1410 g water (70/30 w/w). The spraying is performed with a spraying liquid flow rate of 20 g/min and lasts about 4 hours 15 minutes.

After spraying all of the coating solution, the coated particles are recovered. Their average coating rate is about 50%.

Crushing of the Coated Particles

Approximately 20 g coated particles prepared as described above are crushed for 1 minute using a RM 200 mortar grinder from Retsch, according to the protocol previously described.

All of the powder is recovered and sieved through a nest of sieves having the following decreasing mesh openings: 1.000, 710, 500, 250, 100 and 50 μm and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 5 below.

TABLE 5

Amount of coated
particles (g)

| Size intervals | Intact | Crushed |
|---|---|---|
| 0-50 μm | 0 | 0 |
| 50-100 μm | 0.1 | 0 |
| 100-250 μm | 19.3 | 19.6 |
| 250-500 μm | 1.0 | 0.7 |
| 500-710 μm | 0 | 0 |
| 710-1000 μm | 0 | 0 |
| Average diameter (μm) | 182 | 184 |

| | |
|---|---|
| Change in average | 1.3% |
| diameter | |

The average diameter of the crushed coated particles decreases by 1.3% relative to that of the intact coated particles.

Hence, the coated particles prepared as described above resist crushing.

Dissolution Profiles of the Intact Coated Particles

The in vitro dissolution profile of the intact coated particles prepared as described above is determined by UV spectrometry in 900 ml of 0.1 N HCl maintained at 37.0±0.5° C. and stirred by a paddle rotating at 100 rpm. The obtained dissolution profile is presented in Table 6 below.

TABLE 6

| | % oxycodone |
|---|---|
| Hours | dissolved |
| 0 | 0 |
| 0.25 | 96 |
| 0.5 | 97 |
| 0.75 | 97 |
| 1 | 97 |

After testing for 15 minutes, 96% of the oxycodone dose is dissolved.

The profile therefore exhibits immediate release for the coated particles prepared as described above.

### Example 4

### Preparation of Coated Particles of Oxycodone Hydrochloride Complying with the Invention

Coating Step

300 g granules obtained in Example 1 are coated at room temperature, in a GPCG1.1 fluidized bed apparatus equipped with a Würster tube, with 180.2 g copolymer of butyl methacrylate, 2-dimethylaminoethyl methacrylate and methyl methacrylate (Eudragit® E100 from Evonik) and 120.6 g ethylcellulose (Ethocel® 20 premium from Dow) dissolved in a mixture of 2070 g acetone and 1380 g isopropanol (60/40 w/w). The spraying is performed with a spraying liquid flow rate of 21 g/min.

After spraying all the coating solution, the coated particles are recovered. Their average coating rate is about 50%.

Crushing of the Coated Articles

Approximately 20 g coated particles prepared as described above are crushed for 1 minute using a RM 200 mortar grinder from Retsch, according to the protocol previously described.

All of the powder is recovered and sieved through a nest of sieves having the following decreasing mesh openings: 1,000, 710, 500, 250, 100 and 50 μm and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 7 below.

TABLE 7

Amount of coated
particles (g)

| Size intervals | Intact | Crushed |
|---|---|---|
| 0-50 μm | 0 | 0 |
| 50-100 μm | 0.3 | 0.3 |
| 100-250 μm | 12.2 | 12.0 |
| 250-500 μm | 7.8 | 7.8 |
| 500-710 μm | 0 | 0 |
| 710-1000 μm | 0 | 0 |
| Average diameter (μm) | 250 | 251 |

| | |
|---|---|
| Change in average | 0.4% |
| diameter | |

The average diameter of the crushed coated particles is unchanged relative to that of the intact coated particles.

Hence, the coated particles prepared as described above resist crushing.

Dissolution Profiles of the Intact Coated Particles

The in vitro dissolution profile of the intact coated particles prepared as described above is determined by UV spectrometry in 900 ml 0.1 N HCl maintained at 37.0±0.5° C. and stirred by a paddle rotating at 100 rpm. The obtained dissolution profile is presented in Table 8 below.

TABLE 8

| | % oxycodone |
|---|---|
| Hours | dissolved |
| 0 | 0 |
| 0.25 | 95 |
| 0.5 | 95 |
| 0.75 | 95 |
| 1 | 95 |

After testing for 15 minutes, 95% of the oxycodone dose is dissolved.

The profile therefore exhibits immediate release for the coated particles prepared as described above.

### Example 5

### Preparation of Coated Particles of Morphine Sulphate Complying with the Invention

1615.0 g morphine sulphate and 85.0 g polyvinylpyrrolidone (also named povidone; Plasdone® K29/32 from ISP) are introduced under stirring into a reactor containing 4596.3 g water. The solution is heated to 75° C. When the morphine sulphate crystals and the polyvinylpyrrolidone are dissolved, all of the solution is sprayed onto 300 g cellulose spheres (Cellet 90 from Pharmatrans) in a GPCG1.1 fluidized bed apparatus in a bottom spray configuration. After spraying, the obtained product is sieved through 80 μm and 250 μm sieves. 1700.4 g of 80 μm to 250 μm granules (which corresponds to the fraction of product having passed through the meshes of the 250 μm sieve and being retained on the 80 μm sieve) are then recovered.

Coating Step

300.0 g of obtained granules are coated at room temperature, in a GPCG1.1 fluidized bed apparatus equipped with a Würster tube, with 57.9 g copolymer of butyl methacrylate, 2-dimethylaminoethyl methacrylate and methyl methacrylate (Eudragit® E100 from Evonik), 57.9 g ethylcellulose (Ethocel® 20 premium from Dow) and 12.9 g triethyl citrate (Citrofol AI from Jungbunzlauer) dissolved in a mixture of 887 g acetone and 591 g isoprpanol (60/40 w/w). The spraying is performed with a spraying liquid flow rate of 21 g/min and lasts about 1 hour 30 minutes.

After spraying all the coating solution, the coated particles are recovered. Their average coating rate is about 50%.

Crushing of the Coated Particles

Approximately 20 g coated particles prepared as described above are crushed for 1 minute using a RM 200 mortar grinder from Retsch, according to the protocol previously described.

All of the powder is recovered and sieved through a nest of sieves having the following decreasing mesh openings: 1,000, 710, 500, 250, 100 and 50 μm and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 9 below.

TABLE 9

Amount of coated
particles (g)

| Size intervals | Intact | Crushed |
|---|---|---|
| 0-50 μm | 0 | 0 |
| 50-100 μm | 0.1 | 0.2 |
| 100-250 μm | 20.1 | 19.8 |
| 250-500 μm | 0.2 | 0.3 |
| 500-710 μm | 0 | 0 |
| 710-1000 μm | 0 | 0 |
| Average diameter (μm) | 176 | 177 |

| | |
|---|---|
| Change in average | 0.3% |
| diameter | |

The average diameter of the crushed coated particles is unchanged relative to that of the intact coated particles.

Hence, the coated particles prepared as described above resist crushing.

Dissolution Profiles of the Intact Coated Particles

The in vitro dissolution profile of the intact coated particles prepared as described above is determined by UV spectrometry in 900 ml of 0.1 N HCl maintained at 37.0±0.5<C and stirred by a paddle rotating at 100 rpm. The obtained dissolution profile is presented in Table 10 below.

TABLE 10

| | % morphine |
|---|---|
| Hours | dissolved |
| 0 | 0 |
| 0.5 | 100 |
| 0.75 | 100 |
| 1 | 100 |

After testing for 30 minutes, the entire dose of morphine is dissolved.

The profile therefore exhibits immediate release for the coated particles prepared as described above.

### Example 6

### Preparation of Coated Particles of Hydromorphone Hydrochloride Complying with the Invention

Preparation of the Granules

420.0 g hydromorphone hydrochloride and 280.0 g polyvinylpyrrolidone (also named povidone; Plasdone® K29/32 from ISP) are introduced under stirring into a reactor containing 1050.0 g water. The solution is heated to 70° C. When the hydromorphone hydrochloride crystals and the polyvinylpyrrolidone are dissolved, all of the solution is sprayed onto 1300.0 g cellulose spheres (Cellet® 100 from Pharmatrans) in a GPCG1.1 fluidized bed apparatus in a bottom spray configuration. After spraying, the obtained product is sieved through 80 μm and 250 μm sieves. 1682.2 g of 80 μm to 250 μm granules (which corresponds to the fraction of product having passed through the meshes of the 250 μm sieve and being retained on the 80 μm sieve) are then recovered. 400.0 g granules obtained according to the previous step are coated at room temperature, in a GPCG1.1 fluidized bed apparatus equipped with a Würster tube, with 240.0 g polyvinylpyrrolidone (also named povidone; Plasdone® K29/32 from ISP) and 160 g ethylcellulose (Ethocel® 20 premium from Dow), dissolved in a mixture of 2731 g acetone and 1821 g isopropanol (60/40 (w/w). The spraying is performed with a spraying liquid flow rate of 20 gamin and lasts about 4 hours 5 minutes

After spraying all the solution, the coated particles are recovered. Their average coating rate corresponds to 50%.

Crushing of the Coated Particles

Approximately 20 g coated particles prepared as described above are crushed for 1 minute using a RM 200 mortar grinder from Retsch, according to the protocol previously described.

All of the powder is recovered and sieved through a nest of sieves having the following decreasing mesh openings: 1,000, 710, 500, 250, 100 and 50 μm and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 11 below.

TABLE 11

Amount of coated particles (g)

| Size intervals | Intact | Crushed |
|---|---|---|
| 0-50 μm | 0 | 0.1 |
| 50-100 μm | 0.3 | 0.7 |
| 100-250 μm | 11.5 | 11.4 |
| 250-500 μm | 8.5 | 7.6 |
| 500-710 μm | 0 | 0 |
| 710-1000 μm | 0 | 0 |
| Average diameter (μm) | 178 | 178 |

| | |
|---|---|
| Change in average | 3.8% |
| diameter | |

The average diameter of the crushed coated particles decreases by 3.8% relative to that of the intact coated particles.

Hence, the coated particles prepared as described above resist crushing.

In parallel, around 155 mg coated particles prepared as described above, corresponding to 16 mg dose of hydromorphone hydrochloride, are crushed manually using a 250 ml mortar made in Pyrex and the corresponding pestle also made in Pyrex, for 50 strokes (i.e. 45 seconds).

The recovered powder is observed using a Carl Zeiss Stemi SV 11 type binocular (magnification ×1.6) and is compared to the coated particles prepared as described above and observed before crushing.

The observation of the powder after crushing shows a majority of particles having a size, a shape and a colour similar to those of the coated particles before crushing.

The coated particles prepared as described above resist crushing.

Dissolution Profiles of the Intact Coated Particles

The in vitro dissolution profile of the intact coated particles prepared as described above is determined by UV spectrometry in 900 ml 0.1 N HCl maintained at 37.0±0.5° C. and stirred by a paddle rotating at 100 rpm. The obtained dissolution profile is presented in Table 12 below.

TABLE 12

| | % hydromorphone |
|---|---|
| Hours | dissolved |
| 0 | 0 |
| 0.5 | 99 |
| 0.75 | 99 |
| 1 | 99 |

After testing for 30 minutes, the entire dose of hydromorphone is dissolved.

The profile therefore exhibits immediate release for the coated particles of hydromorphone hydrochloride prepared as described above.

### Example 7

### Preparation of Coated Particles of Hydromorphone Hydrochloride, Complying with the Invention

Coating Step

400.0 g granules obtained according to the previous step are coated at room temperature, in a GPCG1.1 fluidized bed apparatus equipped with a Würster tube, with 320.0 g polyvinylpyrrolidone (also named povidone; Plasdone® K29/32 from ISP) and 80 g ethylcellulose (Ethocel® 20 premium from Dow), dissolved in a mixture of 2731 g acetone and 1821 g isopropanol (60/40 w/w). The spraying is performed with a spraying liquid flow rate of 20 g/min and lasts around 4 hours.

After spraying all of the coating solution, the coated particles are recovered. Their average coating rate is about 50%.

Crushing of the Coated Particles

Approximately 20 g coated particles prepared as described above are crushed for 1 minute using a RM 200 mortar grinder from Retsch, according to the protocol previously described.

All of the powder is recovered and sieved through a nest of sieves having the following decreasing mesh openings: 1,000, 710, 500, 250, 100 and 50 μm and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 13 below.

TABLE 13

Amount of coated particles (g)

| Size intervals | Intact | Crushed |
|---|---|---|
| 0-50 μm | 0 | 1.1 |
| 50-100 μm | 0 | 1.4 |
| 100-250 μm | 10.0 | 10.5 |
| 250-500 μm | 10.0 | 6.9 |
| 500-710 μm | 0 | 0 |
| 710-1000 μm | 0 | 0 |
| Average diameter (μm) | 275 | 229 |

| | |
|---|---|
| Change in average | 16.7% |
| diameter | |

The average diameter of the crushed coated particles decreases by 16.7% relative to that of the intact coated particles.

Hence, the coated particles prepared as described above do not resist crushing if the calculated change in average diameter is considered, in absolute value, less than or equal to 15%, meaning that this change is in the same scale as the one shown by the particles illustrated in the previous examples 1 to 6.

In contrast, regarding a calculated change in average diameter, in absolute value, less than or equal to 20%, the coated particles prepared as described above resist crushing.

Therefore, the particles prepared according to Example 7 exhibit a resistance to crushing lower than that of the particles according Examples 1 to 6, said resistance nevertheless being appropriate to the expected anti-misuse properties in accordance with the invention.

Dissolution Profiles of the Intact Coated Particles

The in vitro dissolution profile of the intact particles prepared as described above is determined by UV spectrometry in 900 ml 0.1 N HCl maintained at 37.0±0.5° C. and stirred by a paddle rotating at 100 rpm. The obtained dissolution profile is presented in Table 14 below.

TABLE 14

| | % hydromorphone |
|---|---|
| Hours | dissolved |
| 0 | 0 |
| 0.5 | 98 |
| 0.75 | 99 |
| 1 | 99 |

After testing for 30 minutes, the entire dose of hydromorphone is dissolved.

The profile therefore exhibits immediate release for the coated particles of hydromorphone hydrochloride as described prepared above.

### Example 8

### Preparation of Coated Particles of Hydromorphone Hydrochloride not in Compliance with the Invention

Preparation of the Granules

315.0 g hydromorphone hydrochloride and 210.0 g polyvinylpyrrolidone (also named povidone; Plasdone® K29/32 from ISP) are introduced under stirring into a reactor containing 787.5 g water. The solution is heated to 70° C. When the hydromorphone hydrochloride crystals and the polyvinylpyrrolidone are dissolved, all of the solution is sprayed onto 975.0 g cellulose spheres (Cellet® 100 from Pharmatrans) in a GPCG1.1 fluidized bed apparatus in a bottom spray configuration. After spraying, the obtained product is sieved through 80 μm and 250 μm sieves. 1332.7 g of 80 μm to 250 μm granules (which corresponds to the fraction of product having passed through the meshes of the 250 φm sieve and being retained on the 80 μm sieve) are then recovered.

Coating Step

380.0 g granules obtained according to the previous step are coated at room temperature, in a GPCG1.1 fluidized bed apparatus equipped with a Würster tube, with 133.0 g hydroxypropylcellulose (Klucel® EF d′Hercules Aqualon) and 247.0 g ethylcellulose (Ethocel® 20 premium from Dow), dissolved in a mixture of 2235 g acetone, 1557 g isopropanol and 432 g water (53/37/10 w/w). The spraying is performed with a spraying liquid flow rate of 20 g/min.

After spraying all of the coating solution, the coated particles are sieved on a 200 μm sieve. The coated particles, greater than 200 μm are recovered. Their average coating rate is about 50%.

Crushing of the Coated Particles

Approximately 20 g coated particles prepared as described above are crushed for 1 minute using a RM 200 mortar grinder from Retsch, according to the protocol previously described.

All of the powder is recovered and sieved through a nest of sieves having the following decreasing mesh openings: 1,000, 710, 500, 250, 100 and 50 μm and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 15 below.

TABLE 15

Amount of coated particles (g)

| Size intervals | Intact | Crushed |
|---|---|---|
| 0-50 μm | 0 | 0 |
| 50-100 μm | 0 | 0 |
| 100-250 μm | 6.6 | 7.0 |
| 250-500 μm | 13.5 | 12.8 |
| 500-710 μm | 0.2 | 0 |
| 710-1000 μm | 0 | 0 |
| Average diameter (μm) | 309 | 304 |

| | |
|---|---|
| Change in average | 1.6% |
| diameter | |

The average diameter of the crushed coated particles decreases by 1.6% relative to that of the intact coated particles.

Hence, the coated particles prepared as described above resist crushing.

Dissolution Profiles of the Intact Coated Particles

The in vitro dissolution profile of the intact coated particles prepared as described above is determined by UV spectrometry in 900 ml 0.1 N HCl maintained at 37.0±0.5° C. and stirred by a paddle rotating at 100 rpm. The obtained dissolution profile is presented in Table 16 below.

TABLE 16

| | % hydromorphone |
|---|---|
| Hours | dissolved |
| 0 | 0 |
| 0.5 | 44 |
| 0.75 | 59 |
| 1 | 70 |
| 1.5 | 86 |
| 2 | 94 |
| 3 | 98 |

Only 44% of the hydromorphone dose is dissolved after testing fir 30 minutes, and only 70% after one hour.

The profile therefore exhibits prolonged release for the coated particles of hydromorphone hydrochloride prepared as described above, release not complying with the invention.

### Example 9

### Preparation of Coated Particles of Hydromorphone Hydrochloride Complying with the Invention

Coating Step

300.0 g granules obtained according to Example 6 (Step 1) are coated at room temperature, in a GPCG1.1 fluidized bed apparatus equipped with a Würster tube, with 150.0 g polyvinylpyrrolidone (also named povidone; Plasdone® K29/32 from ISP), 120 g ammonio methacrylate copolymer (Eudragit® RL 100 from Evonik) and 30.0 g triethylcitrate (Citrofol AI from Jungbunzlauer), dissolved in a mixture of 2070 g acetone and 1380 g isopropanol (60/40 w/w). The spraying is performed with a spraying liquid flow rate of 20 g/imin and lasts about 3 hours 30 minutes

After spraying all the solution, the coated particles are recovered. Their average coating rate corresponds to 50%.

Crushing of the Coated Particles

Approximately 20 g coated particles prepared as described above are crushed for 1 minute using a RM 200 mortar grinder from Retsch, according to the protocol previously described.

All of the powder is recovered and sieved through a nest of sieves having the following decreasing mesh openings: 1,000, 710, 500, 250, 100 and 50 μm and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 17 below.

TABLE 17

Amount of coated particles (g)

| Size intervals | Intact | Crushed |
|---|---|---|
| 0-50 μm | 0 | 0.1 |
| 50-100 μm | 0 | 0.6 |
| 100-250 μm | 17.9 | 18.4 |
| 250-500 μm | 2.4 | 1.6 |
| 500-710 μm | 0 | 0 |
| 710-1000 μm | 0 | 0 |
| Average diameter (μm) | 198 | 187 |

| | |
|---|---|
| Change in average | 5.8% |
| diameter | |

The average diameter of the crushed coated particles decreases by 5.8% relative to that of the intact coated particles.

Hence, the coated particles prepared as described above resist crushing.

Dissolution Profiles of the Intact Coated Particles

The in vitro dissolution profile of the intact coated particles prepared as described above is determined by UV spectrometry in 900 ml 0.1 N HCl maintained at 37.0±0.5° C. and stirred by a paddle rotating at 100 rpm. The obtained dissolution profile is presented in Table 18 below.

TABLE 18

| | % hydromorphone |
|---|---|
| Hours | dissolved |
| 0 | 0 |
| 0.5 | 98 |
| 0.75 | 99 |
| 1 | 99 |

After testing for 30 minutes, the entire dose of hydromorphone is dissolved.

The profile therefore exhibits immediate release for the coated particles of hydromorphone hydrochloride prepared as described above.

### Example 10

### Preparation of Hydromorphone Hydrochloride Capsules Complying with the Invention

Capsules Preparation

175.0 g coated microparticles of hydromorphone hydrochloride prepared as described in Example 6 (Step 2) and 23.0 g polyoxyethylene (Sentry Polyox® WSR 303 NF-LEO from Dow) are blended for 15 minutes using an automatic drum hoop mixer. Then, 2.0 g magnesium stearate are added. The blend is homogenized for 15 minutes using the automatic drum hoop mixer.

The blend is introduced in gelatin size 3 capsules, such that each capsule comprises around 175 mg blend, corresponding to 16 mg hydromorphone hydrochloride.

Dissolution Profile of the Capsules

The in vitro dissolution profile of the capsules prepared as described above is determined by UV spectrometry in 900 ml 0.1 N HCl maintained at 37.0±0.5° C. and stirred by a paddle rotating at 100 rpm. The obtained dissolution profile is presented in Table 19 below.

TABLE 19

| | % hydromorphone |
|---|---|
| Hours | dissolved |
| 0 | 0 |
| 0.5 | 86 |
| 0.75 | 99 |
| 1 | 100 |

After testing for 45 minutes, the entire dose of hydromorphone is dissolved.

The profile therefore exhibits immediate release for the hydromorphone hydrochloride capsules prepared as described above.

In Vitro Test of Extraction in View of Infection

The content of one capsules prepared as described above is crushed for 1 minute using a 250 ml mortar made in Pyrex and the corresponding Pyrex pestle 0.10 ml tap water are added in the mortar containing the crushed powder. The dispersion is stirred using a magnetic stirrer and a magnetic bar (having a length of 2.5 cm) for 10 minutes at room temperature.

The obtained dispersion is observed: it appears as a heterogeneous and viscous liquid.

Then, the obtained dispersion is extracted for 5 minutes using a 10 ml syringe equipped with a 27G needle the tip of which is covered with cotton wool.

The amount of extracted liquid in the syringe is about 0.2 ml, corresponding to about 2% of the introduced extraction solvent volume.

Hence, the capsules prepared as described above comply with the invention.

### Example 11

### Preparation of Hydromorphone Hydrochloride Capsules Complying with the Invention

Capsules Preparation

175.0 g coated microparticles of hydromorphone hydrochloride prepared as described in Example 7 and 23.0 g polyoxyethylene (Sentry Polyox® WSR 303 NF-LEO from Dow) are blended for 15 minutes using an automatic drum hoop mixer. Then, 2.0 g magnesium stearate are added. The blend is homogenized for 15 minutes using the automatic drum hoop mixer.

The blend is introduced in gelatin size 3 capsules, such that each capsule comprises around 174 mg blend, corresponding to 16 mg hydromorphone hydrochloride.

Dissolution Profiles of the Capsules

The in vitro dissolution profile of the capsules prepared as described above is determined by UV spectrometry in 900 ml 0.1 N HCl maintained at 37.0±0.5° C. and stirred by a paddle rotating at 100 rpm. The obtained dissolution profile is presented in Table 21 below.

TABLE 21

| | % hydromorphone |
|---|---|
| Hours | dissolved |
| 0 | 0 |
| 0.5 | 98 |
| 0.75 | 100 |
| 1 | 100 |

After testing for 30 minutes, the entire dose of hydromorphone is dissolved.

The profile therefore exhibits immediate release for the hydromorphone hydrochloride capsules prepared as described above.

Hence, the hydromorphone hydrochloride capsules prepared as described above comply with the invention.

### Example 12

### Preparation of Hydromorphone Hydrochloride Tablets Complying with the Invention

Tablets Preparation

74.5 g coated microparticles of hydromorphone hydrochloride prepared as described in Example 6 (Step 2) are mixed with 9.6 g polyoxyethylene (Sentry Polyox® WSR 303 NF-LEO from Dow), 50.4 g cellulose microcrystalline (Avicel® PH 101 from FMC), 62.6 g mannitol (Pearlitol® SD 200 from Roquette), 1.0 g anhydrous colloidal silica (Aerosil® 200) and 2.0 g magnesium stearate. This blend is used for manufacturing round tablets with a 10 mm diameter and weighting 451 mg, corresponding to 16 mg hydromorphone hydrochloride, using an XP1 tablet press from Korsch. The compression force applied onto the blend is 35 kN. The resulting manufactured tablets have a hardness around 86 N.

### Dissolution Profiles of the Tablets

The in vitro dissolution profile of the tablets prepared as described above is determined by UV spectrometry in 900 ml 0.1 N HCl maintained at 37.0±0.5° C. and stirred by a paddle rotating at 100 rpm. The obtained dissolution profile is presented in Table 23 below.

TABLE 23

| | % hydromorphone |
|---|---|
| Hours | dissolved |
| 0 | 0 |
| 0.5 | 67 |
| 0.75 | 81 |
| 1 | 95 |
| 1.5 | 100 |
| 2 | 100 |

After testing for 45 minutes, more than 75% hydromorphone dose are dissolved.

The profile therefore exhibits immediate release for the hydromorphone hydrochloride tablets prepared as described above.

In Vitro Test of Extraction in View of Injection

One tablet prepared as described above is crushed for 1 minute using a 250 ml mortar made in Pyrex and the corresponding Pyrex pestle. 10 ml tap water are added in the mortar containing the crushed powder. The dispersion is stirred using a magnetic stirrer and a magnetic bar (having a length of 2.5 cm) for 10 minutes at room temperature.

The obtained dispersion is observed: it appears as a heterogeneous and viscous liquid.

Then the obtained dispersion is extracted for 5 minutes using a 10 ml syringe equipped with a 27G needle the tip of which is covered with cotton wool.

The amount of extracted liquid in the syringe is about 0.1 ml, corresponding to about 1% of the introduced extraction solvent volume.

Hence, the hydromorphone hydrochloride tablets prepared as described above comply with the invention.

### Example 13

### Preparation of Oxycodone Hydrochloride Capsules Complying with the Intention

Capsules Preparation

141.4 g coated microparticles of oxycodone hydrochloride prepared according Example 4 (Step 2), 7.1 g polyoxyethylene (Sentry Polyox® WSR 303 NF-LEO from Dow), 35.4 g hydroxypropylcellulose (Klucel® HF from Aqualon-Hercules), 14.1 g xanthan gum (Xantural®@180 from CP Kelco) are blended for 15 minutes using an automatic drum hoop mixer. 2.1 g magnesium stearate are added. The blend is homogenized for 15 minutes using the automatic drum hoop mixer.

The blend is introduced in gelatin size 3 capsules, such that each capsule comprises around 141 mg blend, corresponding to 40 mg oxycodone hydrochloride.

Dissolution Profiles of the Capsules

The in vitro dissolution profile of the capsules prepared as described above is determined by UV spectrometry in 900 ml 0.1 N HCl maintained at 37.0±0.5° C. and stirred by a paddle rotating at 100 rpm. The obtained dissolution profile is presented in Table 24 below.

TABLE 24

| | % hydromorphone |
|---|---|
| Hours | dissolved |
| 0 | 0 |
| 0.5 | 77 |
| 1 | 98 |
| 1.5 | 99 |
| 2 | 99 |

After testing for 30 minutes, more than 75% oxycodone dose are dissolved.

The profile therefore exhibits immediate release for the oxycodone hydrochloride capsules prepared as described above.

In Vitro Test of Extraction in View of Injection

The content of one capsule prepared as described above is crushed for 1 minute using a 250 ml mortar made in Pyrex and the corresponding Pyrex pestle. 10 ml tap water are added in the mortar containing the crushed powder. The dispersion is stirred using a magnetic stirrer and a magnetic bar (having a length of 2.5 cm) for 10 minutes at room temperature.

The obtained dispersion is observed: it appears as a heterogeneous and viscous liquid.

Then the obtained dispersion is extracted for 5 minutes using a 10 ml syringe equipped with a 27G needle the tip of which is covered with cotton wool.

The amount of extracted liquid in the syringe is about 0.3 ml, corresponding to about 3% of the introduced extraction solvent volume.

Hence, the oxycodone hydrochloride capsules prepared as described above comply with the invention.

### Example 14

### Preparation of Coated Particles of Morphine Sulphate, not in Compliance with the Invention

Coating Step

300.0 g granules of morphine sulphate prepared as described in Example 5 (step 1) are coated at room temperature, in a GPCG1.1 fluidized bed apparatus equipped with a Würster tube, with 300.2 g polyvinylpyrrolidone having a molar weight around 1 000 000 g/mol (Kollidon® 90F from BASF), dissolved in a mixture of 2070 g acetone and 1380 g isopropanol (60/40 w/w). The targeted theoretical coating rate is 50%.

The spraying is performed with a spraying liquid flow rate of 7 g/min and lasts around 8 hours 15 minutes.

The spraying is very slow and thus hardly fits with an industrial manufacturing process.

If the coating solution comprising polyvinylpyrrolidone having a molar weight around 1 000 000 g/mol is diluted, the spraying liquid flow rate will be increased. However, the spraying time will not decrease since the amount of spraying solution will have been increased. Moreover, diluting the coating solution with the mixture of acetone and isopropanol induces using substantial amounts of organic solvents.

### Example 15

### Preparation of Coated Particles of Oxycodone Hydrochloride not in Compliance with the Invention

Coating Step

400.0 g granules of oxycodone hydrochloride obtained in Example 1 are coated at room temperature, in a GPCG1.1 fluidized bed apparatus equipped with a Würster tube, with 360.0 g polyvinylpyrrolidone (also named povidone: Plasdone® K29/32 from ISP) and 40.0 g polyethylene glycol (Super Refined PEG 400 LQ MH from Croda), dissolved in a mixture of 2760 g acetone and 1840 g isopropanol (60/40 w/w). The spraying is performed with a spraying liquid flow rate of 20 g/min.

After spraying all of the coating solution, the coated particles are recovered. Their average coating rate is about 50%.

The recovered product is observed using a Carl Zeiss Stemi SV 11 type binocular (magnification ×1.6): the recovered product appears as near spherical, cream-coloured particles which are distinct from one another and seem to have a diameter around 200 μm.

Crushing of the Coated Articles

Around 200 mg coated particles prepared as described above, corresponding to 80 mg dose of oxycodone hydrochloride, are crushed manually using a 250 ml mortar made in Pyrex and the corresponding pestle also made in Pyrex, for 50 strokes (i.e. 45 seconds).

The recovered powder is observed using a Carl Zeiss Stemi SV 11 type binocular (magnification ×1.6). The observation shows a majority of fine white powder composed of fine crystals, clear broken pieces of coating and identified cellulose spheres particles.

It can be deduced that most of the coated particles are destroyed after crushing.

### Example 16

### Preparation of Coated Particles of Hydromorphone Hydrochloride not in Compliance with the Invention

Preparation of the Granules

190.0 g hydromorphone hydrochloride and 10.0 g polyvinylpyrrolidone (also named povidone; Plasdone® K29/32 from ISP) are introduced under stirring into a reactor containing 300.0 g water. The solution is heated to 70° C. When the hydromorphone hydrochloride crystals and the polyvinylpyrrolidone are dissolved, all of the solution is sprayed onto 800.0 g cellulose spheres (Celphere® CP203 from Asai Kasei) in a GPCG1.1 fluidized bed apparatus in a bottom spray configuration. After spraying, the obtained product is sieved through 150 μm and 500 μm sieves. 980.2 g of 150 μm to 500 μm granules (which corresponds to the fraction of product having passed through the meshes of the 500 μm sieve and being retained on the 150 μm sieve) are then recovered.

400.0 g granules obtained according to the previous step are coated at room temperature, in a GPCG1.1 fluidized bed apparatus equipped with a Würster tube, with 80.0 g polyvinylpyrrolidone (also named povidone; Plasdone® K29/32 from ISP) and 53.3 g ethylcellulose (Ethocel® 20 premium from Dow), dissolved in a mixture of 920.0 g acetone and 613.3 g isopropanol (60/40 w/w). The spraying is performed with an average spraying liquid flow rate of 20.5 g/min.

After spraying all of the coating solution, the coated particles are recovered. Their average coating rate is about 50%.

Crushing of the Coated Particles

Approximately 20 g coated particles prepared as described above are crushed for 1 minute using a RM 200 mortar grinder from Retsch, according to the protocol previously described.

All of the powder is recovered and sieved through a nest of sieves having the following decreasing mesh openings: 1,000, 710, 500, 250, 100 and 50 m and a sieve base.

In parallel, 20 g intact coated particles are sieved through a nest of sieves having the same mesh openings: 1,000, 710, 500, 250, 100 and 50 μm and a sieve base.

The particle size distributions obtained for the intact and crushed coated particles as well as the respective calculated average diameters are shown in Table 26 below.

TABLE 26

Amount of coated particles (g)

| Size intervals | Intact | Crushed |
|---|---|---|
| 0-50 μm | 0 | 2.9 |
| 50-100 μm | 0 | 1.6 |
| 100-250 μm | 0.4 | 1.9 |
| 250-500 μm | 21.0 | 14.6 |
| 500-710 μm | 0 | 0 |
| 710-1000 μm | 0 | 0 |
| Average diameter (μm) | 371 | 286 |

| | |
|---|---|
| Change in average | 23.0% |
| diameter | |

The average diameter of the crushed coated particles decreases by 23% relative to that of the intact coated particles.

Hence, the coated particles prepared as described above do not resist crushing.

Dissolution Profiles of the Intact Coated Particles

The in vitro dissolution profile of the intact coated particles prepared as described above is determined by UV spectrometry in 900 ml 0.1 N HCl maintained at 37.0±0.5° C. and stirred by a paddle rotating at 100 rpm. The obtained dissolution profile is presented in Table 27 below.

TABLE 27

| | % hydromorphone |
|---|---|
| Hours | dissolved |
| 0 | 0 |
| 0.5 | 98 |
| 0.75 | 99 |
| 1 | 99 |

After testing for 30 minutes, the entire hydromorphone dose is dissolved.

The profile therefore exhibits immediate release for the coated particles of hydromorphone hydrochloride prepared as described above.

1) An oral dosage form for the immediate release of at least one active compound, comprising coated particles, each of said particles consisting of a non-monocrystalline core containing at least said active compound, said core being coated with at least one coating layer comprising:
(A) at least 15% by weight of a polymer selected from the group consisting of ethylcellulose, cellulose acetate, cellulose acetate butyrate, ammonio (meth)acrylate copolymers, polymers and copolymers of (meth)acrylic acid esters, polyvinyl acetate and mixtures thereof; and
(B) at least 40% by weight of a polymer chosen from low molecular weight polyvinylpyrrolidone, low molecular weight hydroxypropyl methylcellulose, low molecular weight hydroxypropyl cellulose, low molecular weight methylcellulose, low molecular weight hydroxyethyl cellulose, hydroxyethyl methylcellulose, maltodextrin, poloxamers, polyethylene glycols having a molecular weight strictly comprised between 3,000 and 20,000 g/mol, polyvinyl alcohols, vinyl pyrrolidone-vinyl acetate copolymers, xanthan gum, acacia gum, carrageenan gum, guar gum, carob gum, agar-agar, copolymers of methylvinyl ether and maleic anhydride or maleic acid, aminoalkyl methacrylate copolymers, copolymers of butyl methacrylate, 2-dimethylaminoethyl methacrylate and methyl methacrylate 1/2/1, polyvinyl acetate diethyl aminoacetates, the polyvinyl aminoacetals, and mixtures thereof;
the weight ratio polymer (B)/polymer (A) being comprised between 85/15 and 50/50;
and said coating layer representing at least 30% by weight of the total weight of said coated particles.

2) The oral dosage form according to claim 1, wherein it releases at least 75% of the active compound within a period of less than or equal to 45 minutes in a 0.1 N hydrochloric acid solution.

3) The oral dosage form according to claim 1, wherein the core of said coated particles is formed by a carrier particle covered with a layer comprising at least said active compound.

4) The oral dosage form according to claim 1, wherein said coated particles have an average diameter comprised between 50 and 600 μm.

5) The oral dosage form according to claim 1, wherein said coating layer represents from 30 to 60% by weight of the total weight of the coated particles.

6) The oral dosage form according to claim 1, wherein said polymer (A) is chosen from ethylcellulose, cellulose acetate and ammonio (meth)acrylate copolymers.

7) The oral dosage form according to claim 1, wherein said polymer (A) is present in a content comprised between 15 and 60% by weight, preferably between 25 and 50% by weight, in particular between 25 and 45% by weight, and still more particularly between 30 and 45% by weight, relative to the total weight of the coating layer of said coated particles.

8) The oral dosage form according to claim 1, wherein said polymer (B) of the coating of said coated particles is chosen from low molecular weight polyvinyl pyrrolidone, low molecular weight hydroxypropyl methylcellulose, low molecular weight hydroxypropyl cellulose and copolymers of butyl methacrylate, 2-dimethyl aminoethyl methacrylate and methyl methacrylate 1/2/1.

9) The oral dosage form according to claim 1, wherein said polymer (B) is present in a content comprised between 40 and 85% by weight, preferably between 40 and 75% by weight, in particular between 45 to 60% by weight, relative to the total weight of the coating layer of said coated particles.

10) The oral dosage form according to claim 1, wherein the weight ratio polymer B/polymer A of the coating of said coated particles is comprised between 75/25 and 50/50, preferably between 70/30 and 50/50, in particular between 60/40 and 50/50.

11) The oral dosage form according to claim 1, wherein the coating layer of said coated particles also comprises at least one plasticizer, in particular chosen from glycerol and its esters, phthalates, citrates, sebacates, adipates, azelates, benzoates, chlorobutanol, polyethylene glycols having a molecular weight of less than or equal to 3,000 g/mol, vegetable oils, fumarates, malates, oxalates, succinates, butyrates, cetyl alcohol esters, malonates, castor oil and mixtures thereof, said plasticizer being preferably chosen from triethyl citrate and polyethylene glycols having a molecular weight of less than or equal to 3,000 g/mol.

12) The oral dosage form according to claim 1, wherein the plasticizer is present in a content less than or equal to 30% by weight, in particular less than or equal to 20% by weight, more particularly less than 15% by weight, in particular comprised between 5 and 15% by weight, relative to the total weight of the coating layer of said coated particles.

13) The oral dosage form according to claim 1 wherein the coating layer of said particles comprises at most 30% by weight filler relative to its total weight, in particular less than 20% by weight, preferably less than 10% by weight filler relative to its total weight, or is even completely free of filler.

14) The oral dosage form according to the previous claim, wherein the filler is chosen from talc.

15) The oral dosage form according to claim 1, wherein the coating of said coated particle is composed of a single coating layer as described according to any one of claims 1 and 5 to 14.

16) The oral dosage form according to claim 1, wherein the coating represents from 30 to 55% by weight relative to the total weight of the coated particles and comprises:
30 to 45% by weight water-insoluble polymer chosen from ethylcellulose or cellulose acetate;
45 to 60% by weight polymer soluble in a 0.1N hydrochloric acid solution chosen from copolymers of butyl methacrylate, 2-dimethylaminoethyl methacrylate and methyl methacrylate 1/2/1; low molecular weight polyvinyl pyrrolidone and low molecular weight hydroxypropyl methylcellulose; and
0 to 15% by weight plasticizer chosen from triethyl citrate and polyethylene glycol having a molecular weight of approximately 400 g/mol.

17) The oral dosage form according to claim 1, wherein the coating represents from 40 to 55% by weight relative to the total weight of the coated particles and comprises:
30 to 45% by weight ethylcellulose;
45 to 60% by weight copolymers of butyl methacrylate, 2-dimethylaminoethyl methacrylate and methyl methacrylate 1/2/1; and
0 to 10% by weight triethyl citrate or polyethylene glycol having a molecular weight of approximately 400 g/mol.

18) The oral dosage form according to claim 1, wherein the coating represents from 40 to 55% by weight relative to the total weight of the coated particles and comprises:
30 to 45% by weight ethylcellulose;
45 to 60% by weight low molecular weight polyvinyl pyrrolidone; and
0 to 10% by weight triethyl citrate or polyethylene glycol having a molecular weight of approximately 400 g/mol.

19) The oral dosage form according to claim 1, wherein coating layer of said coated particles is obtained by spraying, in particular in a fluidized bed apparatus, a solution, suspension or dispersion containing at least one polymer (A), at least one polymer (B) and optionally at least one plasticizer, onto the cores comprising the active compound.

20) The oral dosage form according to claim 1, wherein it consists in a tablet, a sachet or a capsule.

21) The oral dosage form according to claim 1, comprising, in addition to the coated particles for the immediate release of active compound, at least one viscosifying agent, preferably entirely distinct from coated particles for the immediate release of active compound.

22) The oral dosage form according to claim 1, wherein said active compound is chosen from psychotropics and narcotics, preferably chosen from oxybate, its pharmaceutically acceptable salts, polymorphs and solvates, and opioids and opioid analogues which are more preferably chosen from oxycodone, oxymorphone, hydromorphone, hydrocodone, tramadol, morphine, buprenorphine, dextropropoxyphene, propoxyphene, codeine, fentanyl, alfentanyl, remifentanyl, methadone, pethydine, nalbuphine, levomethadyl acetate, difenoxine, diphenoxylate, loperamide, pentazocine, butorphanol, levorphanol, tapentadol and their pharmaceutically acceptable salts, polymorphs and solvates, more particularly chosen from oxycodone hydrochloride, hydromorphone hydrochloride, oxymorphone hydrochloride or morphine sulphate, and their pharmaceutically acceptable salts, polymorphs and solvates.

23) (canceled)

24) (canceled)

25) (canceled)

26) (canceled)

27) The oral dosage form according to claim 1, wherein said coated particles have an average diameter comprised between 100 and 400 μm.

28) The oral dosage form according to claim 1, wherein said coated particles have an average diameter comprised between 150 and 300 μm.

29) The oral dosage form according to claim 1, wherein said coating layer represents from 30 to 55% by weight of the total weight of the coated particles.

30) The oral dosage form according to claim 1, wherein said coating layer represents from 30 to 50% by weight of the total weight of the coated particles.

31) A method of making a capsule or a sachet, comprising combining coated particles with one or more pharmaceutically acceptable excipients and distributing said coated particles and excipients into capsules or sachets, in sequence or simultaneously, wherein said coated particles consist of a non-monocrystalline core containing an active ingredient said core being coated with at least one coating layer comprising:
(A) at least 15% by weight of a polymer selected from the group consisting of ethylcellulose, cellulose acetate, cellulose acetate butyrate, ammonio (meth)acrylate copolymers, polymers and copolymers of (meth)acrylic acid esters, polyvinyl acetate and mixtures thereof; and
(B) at least 40% by weight of a polymer chosen from low molecular weight polyvinylpyrrolidone, low molecular weight hydroxypropyl methylcellulose, low molecular weight hydroxypropyl cellulose, low molecular weight methylcellulose, low molecular weight hydroxyethyl cellulose, hydroxyethyl methylcellulose, maltodextrin, poloxamers, polyethylene glycols having a molecular weight strictly comprised between 3,000 and 20,000 g/mol, polyvinyl alcohols, vinyl pyrrolidone-vinyl acetate copolymers, xanthan gum, acacia gum, carrageenan gum, guar gum, carob gum, agar-agar, copolymers of methylvinyl ether and maleic anhydride or maleic acid, aminoalkyl methacrylate copolymers, copolymers of butyl methacrylate, 2-dimethylaminoethyl methacrylate and methyl methacrylate 1/2/1, polyvinyl acetate diethyl aminoacetates, polyvinyl aminoacetals, and mixtures thereof;
the weight ratio polymer (B)/polymer (A) being comprised between 85/15 and 50/50; and
said coating layer representing at least 30% by weight of the total weight of said coated particles.

32) The method of claim 31 wherein the core of said coated particles is formed by a carrier particle covered with a layer comprising at least said active compound.

33) The method of claim 31 wherein said coating layer represents from 30 to 60% by weight of the total weight of the coated particles.

34) A method of making a tablet comprising combining coated particles with one or more pharmaceutically acceptable excipients to form a mixture and compressing said mixture into tablets, wherein said coated particles consist of a non-monocrystalline core containing an active ingredient said core being coated with at least one coating layer comprising:
(C) at least 15% by weight of a polymer selected from the group consisting of ethylcellulose, cellulose acetate, cellulose acetate butyrate, ammonio (meth)acrylate copolymers, polymers and copolymers of (meth)acrylic acid esters, polyvinyl acetate and mixtures thereof; and
(D) at least 40% by weight of a polymer chosen from low molecular weight polyvinylpyrrolidone, low molecular weight hydroxypropyl methylcellulose, low molecular weight hydroxypropyl cellulose, low molecular weight methylcellulose, low molecular weight hydroxyethyl cellulose, hydroxyethyl methylcellulose, maltodextrin, poloxamers, polyethylene glycols having a molecular weight strictly comprised between 3,000 and 20,000 g/mol, polyvinyl alcohols, vinyl pyrrolidone-vinyl acetate copolymers, xanthan gum, acacia gum, carrageenan gum, guar gum, carob gum, agar-agar, copolymers of methylvinyl ether and maleic anhydride or maleic acid, aminoalkyl methacrylate copolymers, copolymers of butyl methacrylate, 2-dimethylaminoethyl methacrylate and methyl methacrylate 1/2/1, polyvinyl acetate diethyl aminoacetates, polyvinyl aminoacetals, and mixtures thereof;
the weight ratio polymer (B)/polymer (A) being comprised between 85/15 and 50/50; and
said coating layer representing at least 30% by weight of the total weight of said coated particles.

35) The method of claim 34 wherein the core of said coated particles is formed by a carrier particle covered with a layer comprising at least said active compound.

36) The method of claim 34 wherein said coating layer represents from 30 to 60% by weight of the total weight of the coated particles.
